(19) United States
(12) Patent Application Publication
(10) Pub. No.: US 2015/0376254 A1
(43) Pub. Date: Dec. 31, 2015

(54) GAMMA-MSH ANALOGUES

(71) Applicant: TXP Pharma GmbH, Stans (CH)

(72) Inventor: Thomas Boesen, Copenhagen (DK)

(21) Appl. No.: 14/693,796

(22) Filed: Apr. 22, 2015

Related U.S. Application Data
(60) Provisional application No. 61/982,714, filed on Apr. 22, 2014.

(51) Int. Cl. C07K 14/575

(52) U.S. Cl. CPC C07K 14/575

(57) ABSTRACT

The present invention provides peptide analogues of γ-MSH, comprising the amino acid sequence of human γ-MSH, or variants thereof, and having one or two linear amino acid probe(s) in the N- and/or C-terminal part of the peptide.

### CROSS-REFERENCE

This application claims the benefit of U.S. Provisional Application No. 61/982,714, filed Apr. 22, 2014, which is herein incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Jul. 6, 2015, is named 32103-740-201-Sequence-listing.txt and is 11,884 bytes in size.

### FIELD OF INVENTION

The present invention relates to peptide analogues of the natural existing or native melanocortin γ-melanocyte-stimulating hormone (γ-MSH), or variants thereof, which are modified by N- and/or C-terminal addition of one or two linear amino acid probes, and their use in the treatment of inflammatory and/or ischemic conditions.

### BACKGROUND OF THE INVENTION

The native peptide gamma-melanocyte-stimulating hormone (γ-MSH) is a native agonist for at least a subset of the melanocortin receptors (MCr's). The MCr's belong to the class of G-protein coupled receptors. All receptor subtypes are coupled to a G-stimulatory protein, which means that receptor stimulation involves increased production of cAMP.

The selectivity for the MCr's to bind different MSH peptides varies; the binding affinity of γ-MSH against the MC1r and MC5r is weak, the binding to the MC4r somewhat better, and yet higher affinity to the MC3r (J. Med. Chem. 2005, 48, 1839-1848). Consequently γ-MSH shows some selectivity against the MC3r.

The type 1 (MC1r) and/or type 3 (MC3r) melanocortin receptors are expressed in immune competent cells including monocytes, macrophages, neutrophils, t-cells and dendritic cells. Stimulation of the MCr1 and/or MC3r is associated with modulation of an inflammatory response including attenuation of cytokine production and activation of pro-resolving effects.

Both hypoxia (ischemia) and reperfusion injuries are important factors in human pathophysiology. Examples of tissue hypoxia that predispose to injury during reperfusion include circulatory shock, myocardial ischemia, stroke, temporary renal ischemia, major surgery and organ-transplantation. Because diseases due to ischemia are exceedingly common causes of morbidity and mortality and because organ transplantation is increasingly frequent, treatment strategies with the potential of limiting reperfusion injuries is of great need in order to improve public health.

The underlying pathophysiology of ischemia/reperfusion injuries is complex and involves not only a classical inflammatory reperfusion response with neutrophil-infiltration, but also cytokine gene expression including tumor necrosis factor-α (TNF-α), interleukin (IL)-1β, IL-6, IL-8, interferon-γ, and intercellular adhesion molecule-1 (ICAM-1) within the reperfusion tissue/organ. Furthermore, it has been suggested that locally produced TNF-α contributes to post-ischemic organ dysfunction as in the post-infarctional heart by direct depression of contractility and induction of apoptosis.

Because of the complex nature of ischemia and/or reperfusion injuries simple anti-inflammatory treatment concepts have been shown ineffective. Most experimental studies therefore point to the fact that concomitant interaction with more than one of the activated pathways is needed in order to protect against reperfusion injuries.

Melanocortins have been shown to have both anti-inflammatory, anti-oxidative and anti-apoptotic abilities, and to stimulate pro-resolving effects such as the macrophages ability to phagocytise apoptotic neutrophils. Treatment with the native hormones or known analogues thereof has shown some beneficial effects in animal models of ischemia/reperfusion and inflammatory induced organ failure.

Known analogues of γ-MSH include one or two amino acids in the D-conformation (D-stereoisomer) (see e.g. Grieco et al., J Med Chem 2000; 43:4998-5002).

### SUMMARY OF THE INVENTION

The present invention provides peptide analogues of γ-MSH comprising the amino acid sequence of γ-MSH, preferably human γ1- or γ2-MSH, or specified variants thereof, and one or two linear amino acid probes covalently bound to the N- and/or C-terminus of said γ-MSH. These are collectively referred to herein as γ-MSH analogues.

In some embodiments, the γ-MSH analogues provided herein have one or more improved properties compared to the native γ-MSH peptide. For example, in some embodiments, the γ-MSH analogues provided herein have improved binding to one or more of the melanocortin receptors, such as MC1r and/or MC3r. In some embodiments, the γ-MSH analogues provided herein have improved activation of one more of the melanocortin receptors, such as MC1r and/or MC3r. For example, in some embodiments, the γ-MSH analogues provided herein have improved stability and/or reduced propensity for degradation by proteases.

Thus, the present invention relates to a γ-MSH analogue being a peptide consisting of from 8 to 52 amino acids, said peptide comprising the amino acid sequence:

{X₁}-(Aa₁)ₙ-Y-(Aa₂)ₘ-Z-{X₂}

wherein n is a number selected from 0, 1, 2, 3 and 4, and (aa₁) independently is any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) is any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein {X₁} is an optional linear amino acid probe consisting of from 2 to 20 consecutive amino acid residues covalently linked to (aa₁)ₙ,
wherein {X₂} is an optional linear amino acid probe consisting of from 2 to 20 consecutive amino acid residues covalently linked to Z,
with the proviso that {X₁}, or {X₂}, or {X₁} and {X₂}, is present.

In one embodiment, (aa₁)ₙ is a sequence consisting of 4 contiguous amino acids (n=4), selected from Tyr-Val-Met-Gly (SEQ ID NO: 2) and Tyr-Val-Nle-Gly; and in one embodiment (aa₂)ₘ is 1 amino acid (m=1), such as Asp.

In one embodiment said one or two linear amino acid probes individually consist of 2 to 20 consecutive amino acid residues, for example 3 to 10, such as 4 to 8 consecutive amino acid residues.

In one embodiment said amino acid residues of said one or two linear amino acid probes are individually selected from any proteinogenic amino acid or non-proteinogenic amino acid, in one particular embodiment selected from the group consisting of Lys, (D-Lys), Glu and (D-Glu).

The present invention also encompasses pharmaceutical compositions comprising the γ-MSH analogues of the present invention, as well as the γ-MSH analogues of the present invention for use as a medicament.

In one embodiment the γ-MSH analogues according to the present invention are suitable for use in the treatment of an ischemic and/or inflammatory condition in the tissue of one or more organs of a mammal.

### DETAILED DESCRIPTION OF THE INVENTION

Proopiomelanocortin (POMC) is a precursor polypeptide which is cleaved enzymatically into a range of peptides, including the melanocortins. The melanocortins include adrenocorticotropic hormone (ACTH) and the different forms of melanocyte-stimulating hormone (MSH): α-MSH, β-MSH and γ-MSH. They exert their effects by binding to and activating the melanocortin receptors MC1r to MC5r, each with differing specificities for the melanocortins.

Three forms of γ-melanocyte-stimulating hormone or γ-melanotropin (γ-MSH) exist namely γ1-MSH, γ2-MSH and γ3-MSH, which differ in the structure of their C-termini. γ1-MSH and γ2-MSH vary by one amino acid in the C-terminus.

### γ1-MSH Tyr-Val-Met-Gly-His-Phe-Arg-Trp-Asp-Arg-Phe-Gly (SEQ ID NO: 89)

YVMGHFRWDRFG
P01189[77-88], Pro-opiomelanocortin, Homo sapiens
aa modifications: Phenylalanine amide (pos 88)

### γ2-MSH Tyr-Val-Met-Gly-His-Phe-Arg-Trp-Asp-Arg-Phe (SEQ ID NO: 90)

YVMGHFRWDRF
P01189[77-87], Pro-opiomelanocortin, Homo sapiens
aa modifications: Phenylalanine amide (pos 87)

### Analogues of γ-MSH

It is an aspect of the present invention to provide γ-MSH peptide analogues. The γ-MSH peptide analogues in one embodiment comprise the amino acid sequence of γ-MSH, in one embodiment human γ-MSH, such as human γ1- or γ2-MSH, or variants thereof, and further comprise one or two linear amino acid probes.

In one embodiment the γ-MSH peptide, or variants thereof, and the one or two linear amino acid probes are covalently bound or linked together by peptide bond(s).

In one embodiment the one or two linear amino acid probes are covalently bound to the N-terminus and/or the C-terminus of the γ-MSH peptide, or variants thereof.

In some embodiments, the γ-MSH analogues provided herein have certain improved properties, for instance with respect to binding affinity and/or activation of one or two melanocortin receptors, such as MC1r and/or MC3r. Still further, in another embodiment, the γ-MSH analogues provided herein are more stable, such as less susceptible to proteases.

The γ-MSH analogues of the present invention in one embodiment comprises all or part of the amino acid sequence of human γ-MSH (preferably γ1 or γ2), or variants thereof, and one or two linear amino acid probes.

‘X’ is used herein to refer to a linear amino acid probe. ‘X₁’ is used to specify that the linear amino acid probe, or X, is covalently bound to the most N-terminal part of the γ-MSH peptide. When referred to as {X₁}, a linear amino acid probe is optionally comprised in the N-terminal part of the peptide sequence. ‘X₂’ is used to specify that the linear amino acid probe, or X, is covalently bound to the most C-terminal part of the γ-MSH peptide. When referred to as {X₂}, the linear amino acid probe is optionally comprised in the C-terminal part of the peptide sequence.

It is understood that when referring to a γ-MSH analogue herein, such as a γ-MSH analogue comprising one or two linear amino acid probes, such as comprising one or two linear amino acid probes covalently bound to the N- and/or C-terminus of said γ-MSH peptide, this means that the γ-MSH analogue of the invention may
comprise one linear amino acid probe bound to the N-terminus of γ-MSH, or variants thereof, (X₁),
comprise one linear amino acid probe bound to the C-terminus of γ-MSH, or variants thereof, (X₂), or
comprise two linear amino acid probes bound to the N-terminus, and the C-terminus, respectively, of γ-MSH, or variants thereof (X₁ and X₂).

In one embodiment the γ-MSH analogue of the present invention comprises the amino acid sequence:

(aa₁)ₙ-Y-(aa₂)ₘ-Z

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein said sequence further comprises one or two linear amino acid probes covalently bound to the N- and/or C-terminus of said γ-MSH amino acid sequence.

In one embodiment the γ-MSH analogue of the present invention comprises the amino acid sequence:

{X₁}-(aa₁)ₙ-Y-(aa₁)ₘ-Z-{X₂}

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue; wherein Z comprises an amino acid sequence selected from the group consisting of Arg-Phe-Gly; Arg-(D-Phe)-Gly; Arg-Phe and Arg-(D-Phe); and
wherein {X₁} is an optional linear amino acid probe covalently linked to (aa₁)ₙ,
wherein {X₂} is an optional linear amino acid probe covalently linked to Z,
with the proviso that at least {X₁}, or {X₂}, or {X₁} and {X₂}, is present.

In one embodiment the γ-MSH analogue of the present invention comprises the amino acid sequence:

X₁-(aa₁)ₙ-Y-(aa₂)ₘ-Z

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₁ is a linear amino acid probe covalently linked to (aa₁)ₙ.

In one embodiment the γ-MSH analogue of the present invention comprises the amino acid sequence:

X₁-(aa₁)ₙ-Y-(aa₂)ₘ-Z-X₂

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₁ is a linear amino acid probe covalently linked to (aa₁)ₙ, and X₂ is a linear amino acid probe covalently linked to Z.

In one embodiment the γ-MSH analogue of the present invention comprises the amino acid sequence:

(aa₁)ₙ-Y-(aa₂)ₘ-Z-X₂

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₂ is a linear amino acid probe covalently linked to Z.

‘-’ of the amino acid sequence formulas used herein denotes a peptide bond.

In one embodiment, the most carboxy terminal amino acid of the γ-MSH sequence is amidated. In one embodiment, the most carboxy terminal Gly of the γ-MSH sequence is Glycine amide (—NH₂). In another embodiment, the most carboxy terminal Phe or (D-Phe) of γ-MSH is Phenylalanine amide (—NH₂).

In the embodiments where a linear amino acid probe is bound to the C-terminus of a γ-MSH sequence, the most C-terminal amino acid of said γ-MSH sequence, such as Gly Phe and (D-Phe), may not be amidated.

A natural amino acid is a naturally occurring amino acid existing in nature and being naturally incorporated into polypeptides (proteinogenic amino acid). They consist of the 20 genetically encoded amino acids Ala, Arg, Asn, Asp, Cys, Gln, Glu, Gly, His, Ile, Leu, Lys, Met, Phe, Pro, Ser, Tyr, Thr, Trp, Val, and 2 which are incorporated into proteins by unique synthetic mechanisms: Sec (selenocysteine, or U) and Pyl (pyrrolysine, 0). These are naturally all L-stereoisomers.

Aside from the 22 natural or standard amino acids, there are many other non-naturally occurring amino acids (non-proteinogenic or non-standard amino acids). They are either not found in proteins, or are not produced directly and in isolation by standard cellular machinery.

Non-standard amino acids are usually formed through modifications to standard amino acids, such as post-translational modifications. Examples of preferred unnatural amino acid residues according to the invention are Nle (Norleucine), Orn (ornithine, deguanylated Arginine), Nal (beta-2-naphthyl-alanine), D-Nal (beta-2-naphthyl-D-alanine), D-Arg, D-Trp, D-Phe and D-Val.

Any amino acids according to the present invention may be in the L- or D-configuration. If nothing is specified, reference to the L-isomeric form is preferably meant.

The term peptide also embraces post-translational modifications introduced by chemical or enzyme-catalyzed reactions, as are known in the art. Such post-translational modifications can be introduced prior to partitioning, if desired. Also, functional equivalents may comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

Sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of e.g a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same molecules, including dimers or unrelated chemical moieties. Such functional equivalents are prepared by linkage of functionalities to groups which are found in fragment including at any one or both of the N- and C-termini, by means known in the art.

In some embodiments, the peptides according to the present invention are modified by acetylation of the MSH peptide. In some embodiments the peptides according to the present invention are modified by C-terminal amidation. In one embodiment such modification increases the stability of the peptides.

In one embodiment, the carboxy terminus of said peptide or MSH-analogue as defined herein above is —C(═O)—B1, wherein B1 is selected from OH, NH₂, NHB2, N(B2)(B3), OB2, and B2, and wherein B2 and B3 are independently selected from optionally substituted C₁₋₆ alkyl, optionally substituted C₂₋₆ alkenyl, optionally substituted C₆₋₁₀ aryl, optionally substituted C₇₋₁₆ aralkyl, and optionally substituted C₇₋₁₆ alkylaryl.

In a specific embodiment, the carboxy-terminus of said peptide is —C(═O)—B1, wherein B1 is OH or NH₂.

In one embodiment, the amino terminus of said peptide is (B4)HN—, (B4)(B5)N—, or (B6)HN—, wherein B4 and B5 are independently selected from H, optionally substituted C₁₋₆ alkyl, optionally substituted C₂₋₆ alkenyl, optionally substituted C₆₋₁₀ aryl, optionally substituted C₇₋₁₆ aralkyl, and optionally substituted C₇₋₁₆ alkylaryl; and B6 is B4-C(═O)—.

In another embodiment the amino terminus of said peptide is (B6)HN—, wherein B6 is B4-C(═O)— and B4 is CH₃. In yet another embodiment the amino terminus of said peptide is (B4)HN—, wherein B4 is H.

According to the present invention, the term “optionally substituted” is intended to mean that the group in question may be substituted one or several times, such as 1 to 5 times, preferably 1 to 3 times, most preferably 1 to 2 times, with one or more groups selected from C₁₋₈-alkyl, C₁₋₈-alkoxy, oxo (which may be represented in the tautomeric enol form), carboxyl, amino, hydroxy (which when present in an enol system may be represented in the tautomeric keto form), nitro, cyano, dihalogen-C₁₋₈-alkyl, trihalogen-C₁₋₈-alkyl and halogen. In general, the above substituents may be susceptible to further optional substitution.

According to the present invention, the term C₁₋₆-alkyl is intended to mean a linear or branched saturated hydrocarbon chain wherein the longest chains has from one to six carbon atoms, such as methyl, ethyl, n-propyl, isopropyl, n-butyl, isobutyl, sec-butyl, tert-butyl, pentyl, isopentyl, neopentyl, hexyl, heptyl and octyl. A branched hydrocarbon chain is intended to mean a C₁₋₆-alkyl substituted at any carbon with a hydrocarbon chain.

According to the present invention, the term C₂₋₆-alkenyl is intended to mean a linear or branched hydrocarbon group having from two to six carbon atoms and containing one or more double bonds. Illustrative examples of C₂₋₆-alkenyl groups include allyl, homo-allyl, vinyl, crotyl, butenyl, pentenyl and hexenyl. Illustrative examples of C₂₋₆-alkenyl groups with more than one double bond include butadienyl, pentadienyl, hexadienyl, and hexatrienyl groups as well as branched forms of these. The position of the unsaturation (the double bond) may be at any position along the carbon chain.

According to the present invention, the term C₃₋₈-cycloalkyl is intended to cover three-, four-, five-, six- seven-, and eight-membered rings comprising carbon atoms only whereas the term hetero-cyclyl is intended to mean three-, four-, five-, six- seven-, and eight-membered rings wherein carbon atoms together with from 1 to 3 heteroatoms constitute said ring. The heteroatoms are independently selected from oxygen, sulphur, and nitrogen. C₃₋₈-cycloalkyl and heterocyclyl rings may optionally contain one or more unsaturated bonds.

Illustrative examples of C₃₋₈-cycloalkyl are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene, 1,2-cycloheptadiene, 1,3-cycloheptadiene, 1,4-cycloheptadiene and 1,3,5 cycloheptatriene.

Illustrative examples of heterocyclyls are the heterocycles 2H-thipyran, 3H-thipyran, 4H-thipyran, tetrahydrothiopyran, 2H-pyran, 4H-pyran, tetrahydropyran, piperidine, 1,2-dithiin, 1,2-dithiane, 1,3-dithiin, 1,3-dithiane, 1,4-dithiin, 1,4-dithiane, 1,2-dioxin, 1,2-dioxane, 1,3-dioxin, 1,3-dioxane, 1,4-dioxin, 1,4-dioxane, piperazine, 1,2-oxathiin, 1,2-oxathiane, 4H-1,3-oxathiin, 1,3-oxathiane, 1,4-oxathiin, 1,4-oxathiane, 2H-1,2-thiazine, tetrahydro-1,2-thiazine, 2H-1,3-thiazine, 4H-1,3-thiazine, 5,6-dihydro-4H-thiazine, 4H-1,4-thiazine, tetrahydro-1,4-thiazine, 2H-1,2-oxazine, 4H-1,2-oxazine, 6H-1,2-oxazine, 2H-1,3-oxazine, 4H-1,3-oxazine, 4H-1,4-oxazine, maleimide, succinimide, imidazole, pyrazole, pyrrole, oxazole, furazan, barbituric acid, thiobarbituric acid, dioxopiperazine, isoxazole, hydantoin, dihydrouracil, morpholine, trioxane, 4H-1,2,3-trithiin, 1,2,3-trithiane, 1,3,5-trithiane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,2-dioxole, 1,2-dioxolane, 1,3-dioxole, 1,3-dioxolane, 3H-1,2-dithiole, 1,2-dithiolane, 1,3-dithiole, 1,3-dithiolane, isoxazoline, isoxazolidine, oxazoline, oxazolidine, thiazoline, thiozolidine, 3H-1,2-oxathiole, 1,2-oxathiolane, 5H-1,2-oxathiole, 1,3-oxathiole, 1,3-oxathiolane, 1,2,3-trithiole, 1,2,3-trithiolane, 1,2,4-trithiolane, 1,2,3-trioxole, 1,2,3-trioxolane, 1,2,4-trioxolane, 1,2,3-triazoline and 1,2,3-triazolidine. Binding to the heterocycle may be at the position of the heteroatom or via carbon atom of the heterocycle.

According to the present invention the term aryl is intended to mean a carbocyclic aromatic ring or ring system. Moreover, the term aryl includes fused ring systems wherein at least two aryl rings, or at least one aryl and at least one C₃₋₈-cycloalkyl, or at least one aryl and at least one heterocyclyl, share at least chemical bond. Illustrative examples of aryl rings include optionally substituted phenyl, naphthalenyl, phenanthrenyl, anthracenyl, acenaphthylenyl, tetralinyl, fluorenyl, indenyl, indolyl, coumaranyl, coumarinyl, chromanyl, isochromanyl, and azulenyl. A preferred aryl group is phenyl.

C₇₋₁₆ aralkyl is intended to mean a C₆₋₁₀ aryl substituted with C₁₋₆ alkyl and C₇₋₁₆ alkylaryl is intended to mean a C₁₋₆ alkyl substituted with C₆₋₁₀ aryl.

### Linear Amino Acid Probe

In one embodiment the γ-MSH analogues of the present invention comprises all or part of the amino acid sequence of human γ-MSH (such as γ1- or γ2-MSH), or variants thereof, and one or two linear amino acid probes (also referred to as X). In one embodiment the γ-MSH peptide sequence and each of the one or two linear amino acid probes are covalently bound together by peptide bond(s).

A ‘linear amino acid probe’ is defined herein as a short peptide sequence in linear conformation. Thus the linear amino acid probe comprises a stretch of consecutive amino acid residues, which individual residues are covalently bound or linked together via regular peptide bonds.

In one embodiment of the invention, a linear amino acid probe consists of from 2 to 20 consecutive amino acid residues, which residues are covalently bound or linked together via peptide bonds.

In one embodiment, a linear amino acid probe according to the invention consists of 2 to 20 consecutive amino acid residues, for example 2 to 3, such as 3 to 4, for example 4 to 5, such as 5 to 6, for example 6 to 7, such as 7 to 8, for example 8 to 9, such as 9 to 10, for example 10 to 11, such as 11 to 12, for example 12 to 13, such as 13 to 14, for example 14 to 15, such as 15 to 16, for example 16 to 17, such as 17 to 18, for example 18 to 19, such as 19 to 20 consecutive amino acid residues.

In one embodiment a linear amino acid probe according to the invention consists of 2-3, such as 2-4, for example 2-5, 2-6, 2-7, 2-8, 2-9, such as 2-10 consecutive amino acid residues.

In one embodiment a linear amino acid probe according to the invention consists of 3-4, for example 3-5, 3-6, 3-7, 3-8, 3-9, such as 3-10 consecutive amino acid residues.

In one embodiment a linear amino acid probe according to the invention consists of 4-5, such as 4-6, 4-7, 4-8, 4-9, such as 4-10 consecutive amino acid residues.

In one embodiment a linear amino acid probe according to the invention consists of 5-6, 5-7, 5-8, 5-9, such as 5-10 consecutive amino acid residues.

In one embodiment a linear amino acid probe according to the invention consists of 6-7, 6-8, 6-9, such as 6-10 consecutive amino acid residues.

In one embodiment, a linear amino acid probe according to the invention consists of 2 consecutive amino acid residues, for example 3, such as 4, for example 5, such as 6, for example 7, such as 8, for example 9, such as 10, for example 11, such as 12, for example 13, such as 14, for example 15, such as 16, for example 17, such as 18, for example 19, such as 20 consecutive amino acid residues.

In a specific embodiment a linear amino acid probe according to the invention consists of 3, 4, 5 or 6 consecutive amino acid residues.

The nature of each amino acid of the linear amino acid probe may vary i.e. they may be identical with respect to each other, or they may not be identical with respect to each other. Thus, in one embodiment, each amino acid of the linear amino acid probe are identical with respect to each other. In another embodiment, the amino acids of the linear amino acid probe are at least partly non-identical with respect to each other. For example, a linear probe may comprise more than one Lys, more than one Orn, and/or more than one Glu.

In one embodiment each amino acid of the linear amino acid probe is individually selected from any natural (or proteinogenic) amino acid or a non-naturally occurring (or non-proteinogenic) amino acid.

In one embodiment each amino acid of the linear amino acid probe is individually selected from the group consisting of Ala, Arg, Asn, Asp, Cys, Gln, Glu, Gly, His, Ile, Leu, Lys, Met, Phe, Pro, Ser, Tyr, Thr, Trp, Val, Sec and Pyl.

In one embodiment each amino acid of the linear amino acid probe is individually selected from the group consisting of Ala, Arg, Asn, Asp, Cys, Gln, Glu, Gly, His, Ile, Leu, Lys, Met, Phe, Pro, Ser, Tyr, Thr, Trp, Val, Sec, Pyl and Orn.

Any amino acid according to the present invention may be in the L- or D-configuration. If nothing is specified, reference to the L-isomeric form is preferably meant.

In one embodiment the amino acids of the linear amino acid probe are selected from the group consisting of Lys, D-Lys, L-Lys, Orn, L-Orn, D-Orn, Glu, D-Glu and L-Glu.

In one embodiment the amino acids of the linear amino acid probe are selected from the group consisting of Lys, D-Lys, L-Lys, Glu, D-Glu and L-Glu.

In one embodiment, one, two, three, four, five, six, seven, eight, nine or ten of the amino acids of the linear amino acid probe are individually selected from the group consisting of Lys, D-Lys, L-Lys, Glu, D-Glu and L-Glu.

In a specific embodiment each amino acid of the linear amino acid probe is individually selected from the group consisting of Lys, D-Lys, L-Lys, Glu, D-Glu and L-Glu.

In a specific embodiment each amino acid of the linear amino acid probe is individually selected from the group consisting of Lys L-Lys and (D-Lys).

In a specific embodiment each amino acid of the linear amino acid probe is individually selected from the group consisting of Glu, L-Glu and (D-Glu).

In one embodiment the linear amino acid probe is selected from the group consisting of Lys-Lys (Lys₂); Lys-Lys-Lys (Lys₃); Lys-Lys-Lys-Lys (Lys₄) (SEQ ID NO: 3); Lys-Lys-Lys-Lys-Lys (Lys₅) (SEQ ID NO:4); Lys-Lys-Lys-Lys-Lys-Lys (Lys₆) (SEQ ID NO: 5); Lys-Lys-Lys-Lys-Lys-Lys-Lys (Lys₇) (SEQ ID NO: 6); Lys-Lys-Lys-Lys-Lys-Lys-Lys-Lys (Lys₈) (SEQ ID NO: 7); Lys-Lys-Lys-Lys-Lys-Lys-Lys-Lys-Lys (Lys₉) (SEQ ID NO: 8); Lys-Lys-Lys-Lys-Lys-Lys-Lys-Lys-Lys-Lys (Lys₁₀) (SEQ ID NO: 9); Lys-Lys-Lys-Lys-Lys-Lys-Lys-Lys-Lys-Lys-Lys (Lys₁₁) (SEQ ID NO: 10); Lys-Lys-Lys-Lys-Lys-Lys-Lys-Lys-Lys-Lys-Lys-Lys (Lys₁₂) (SEQ ID NO: 11); Lys-Lys-Lys-Lys-Lys-Lys-Lys-Lys-Lys-Lys-Lys-Lys-Lys (Lys₁₃) (SEQ ID NO: 12); Lys-Lys-Lys-Lys-Lys-Lys-Lys-Lys-Lys-Lys-Lys-Lys-Lys-Lys (Lys₁₄) (SEQ ID NO: 13); and Lys-Lys-Lys-Lys-Lys-Lys-Lys-Lys-Lys-Lys-Lys-Lys-Lys-Lys-Lys (Lys₁₅) (SEQ ID NO: 14), wherein each Lys may individually be selected from L-Lys and D-Lys.

In one embodiment the linear amino acid probe is selected from the group consisting of Glu-Glu (Glu₂); Glu-Glu-Glu (Glu₃); Glu-Glu-Glu-Glu (Glu₄) (SEQ ID NO: 15); Glu-Glu-Glu-Glu-Glu (Glu₅) (SEQ ID NO: 16); Glu-Glu-Glu-Glu-Glu-Glu (Glu₆) (SEQ ID NO: 17); Glu-Glu-Glu-Glu-Glu-Glu-Glu (Glu₇) (SEQ ID NO: 18); Glu-Glu-Glu-Glu-Glu-Glu-Glu-Glu (Glu₈) (SEQ ID NO: 19); Glu-Glu-Glu-Glu-Glu-Glu-Glu-Glu-Glu (Glu₉) (SEQ ID NO: 20); Glu-Glu-Glu-Glu-Glu-Glu-Glu-Glu-Glu-Glu (Glu₁₀) (SEQ ID NO: 21); Glu-Glu-Glu-Glu-Glu-Glu-Glu-Glu-Glu-Glu-Glu (Glu₁₁) (SEQ ID NO: 22); Glu-Glu-Glu-Glu-Glu-Glu-Glu-Glu-Glu-Glu-Glu-Glu (Glu₁₂) (SEQ ID NO: 23); Glu-Glu-Glu-Glu-Glu-Glu-Glu-Glu-Glu-Glu-Glu-Glu-Glu (Glu₁₃) (SEQ ID NO: 24); Glu-Glu-Glu-Glu-Glu-Glu-Glu-Glu-Glu-Glu-Glu-Glu-Glu-Glu (Glu₁₄) (SEQ ID NO: 25) and Glu-Glu-Glu-Glu-Glu-Glu-Glu-Glu-Glu-Glu-Glu-Glu-Glu-Glu-Glu (Glu₁₅) (SEQ ID NO: 26), wherein each Glu may individually be selected from L-Glu and D-Glu.

In one embodiment the linear amino acid probe is selected from the group consisting of Glu-Lys-Lys-Lys-Lys-Lys (SEQ ID NO: 27), Lys-Glu-Lys-Lys-Lys-Lys (SEQ ID NO: 28), Lys-Lys-Glu-Lys-Lys-Lys (SEQ ID NO: 29), Lys-Lys-Lys-Glu-Lys-Lys (SEQ ID NO: 30), Lys-Lys-Lys-Lys-Glu-Lys (SEQ ID NO: 31), Lys-Lys-Lys-Lys-Lys-Glu (SEQ ID NO: 32), Glu-Glu-Lys-Lys-Lys-Lys (SEQ ID NO: 33), Glu-Lys-Glu-Lys-Lys-Lys (SEQ ID NO: 34), Glu-Lys-Lys-Glu-Lys-Lys (SEQ ID NO: 35), Glu-Lys-Lys-Lys-Glu-Lys (SEQ ID NO: 36), Glu-Lys-Lys-Lys-Lys-Glu (SEQ ID NO: 37), Lys-Glu-Glu-Lys-Lys-Lys (SEQ ID NO: 38), Lys-Glu-Lys-Glu-Lys-Lys (SEQ ID NO: 39), Lys-Glu-Lys-Lys-Glu-Lys (SEQ ID NO: 40), Lys-Glu-Lys-Lys-Lys-Glu (SEQ ID NO: 41), Lys-Lys-Glu-Glu-Lys-Lys (SEQ ID NO: 42), Lys-Lys-Glu-Lys-Glu-Lys (SEQ ID NO: 43), Lys-Lys-Glu-Lys-Lys-Glu (SEQ ID NO: 44), Lys-Lys-Lys-Glu-Glu-Lys (SEQ ID NO: 45), Lys-Lys-Lys-Glu-Lys-Glu (SEQ ID NO: 46), Lys-Lys-Lys-Lys-Glu-Glu (SEQ ID NO: 47), Glu-Glu-Glu-Lys-Lys-Lys (SEQ ID NO: 48), Glu-Glu-Lys-Glu-Lys-Lys (SEQ ID NO: 49), Glu-Glu-Lys-Lys-Glu-Lys (SEQ ID NO: 50), Glu-Glu-Lys-Lys-Lys-Glu (SEQ ID NO: 51), Glu-Lys-Glu-Glu-Lys-Lys (SEQ ID NO: 52), Glu-Lys-Glu-Lys-Glu-Lys (SEQ ID NO: 53), Glu-Lys-Glu-Lys-Lys-Glu (SEQ ID NO: 54), Glu-Lys-Lys-Glu-Glu-Lys (SEQ ID NO: 55), Glu-Lys-Lys-Glu-Lys-Glu (SEQ ID NO: 56), Glu-Lys-Lys-Lys-Glu-Glu (SEQ ID NO: 57), Lys-Lys-Lys-Glu-Glu-Glu (SEQ ID NO: 58), Lys-Lys-Glu-Lys-Glu-Glu (SEQ ID NO: 59), Lys-Lys-Glu-Glu-Lys-Glu (SEQ ID NO: 60), Lys-Lys-Glu-Glu-Glu-Lys (SEQ ID NO: 61), Lys-Glu-Lys-Lys-Glu-Glu (SEQ ID NO: 62), Lys-Glu-Lys-Glu-Lys-Glu (SEQ ID NO: 63), Lys-Glu-Lys-Glu-Glu-Lys (SEQ ID NO: 64), Lys-Glu-Glu-Lys-Lys-Glu (SEQ ID NO: 65), Lys-Glu-Glu-Lys-Glu-Lys (SEQ ID NO: 66), Lys-Glu-Glu-Glu-Lys-Lys (SEQ ID NO: 67), Lys-Lys-Glu-Glu-Glu-Glu (SEQ ID NO: 68), Lys-Glu-Lys-Glu-Glu-Glu (SEQ ID NO: 69), Lys-Glu-Glu-Lys-Glu-Glu (SEQ ID NO: 70), Lys-Glu-Glu-Glu-Lys-Glu (SEQ ID NO: 71), Lys-Glu-Glu-Glu-Glu-Lys (SEQ ID NO: 72), Glu-Lys-Lys-Glu-Glu-Glu (SEQ ID NO: 73), Glu-Lys-Glu-Lys-Glu-Glu (SEQ ID NO: 74), Glu-Lys-Glu-Glu-Lys-Glu (SEQ ID NO: 75), Glu-Lys-Glu-Glu-Glu-Lys (SEQ ID NO: 76), Glu-Glu-Lys-Lys-Glu-Glu (SEQ ID NO: 77), Glu-Glu-Lys-Glu-Lys-Glu (SEQ ID NO: 78), Glu-Glu-Lys-Glu-Glu-Lys (SEQ ID NO: 79), Glu-Glu-Glu-Lys-Lys-Glu (SEQ ID NO: 80), Glu-Glu-Glu-Lys-Glu-Lys (SEQ ID NO: 81), Glu-Glu-Glu-Glu-Lys-Lys (SEQ ID NO: 82), Lys-Glu-Glu-Glu-Glu-Glu (SEQ ID NO: 83), Glu-Lys-Glu-Glu-Glu-Glu (SEQ ID NO: 84), Glu-Glu-Lys-Glu-Glu-Glu (SEQ ID NO: 85), Glu-Glu-Glu-Lys-Glu-Glu (SEQ ID NO: 86), Glu-Glu-Glu-Glu-Lys-Glu (SEQ ID NO: 87), Glu-Glu-Glu-Glu-Glu-Lys (SEQ ID NO: 88), Glu-Glu-Glu-Glu-Glu-Glu (SEQ ID NO: 17).

### Length of γ-MSH Analogues

It is an aspect of the invention to provide a peptide consisting of from 8 to 52 amino acids, said peptide comprising the amino acid sequence of γ-MSH (preferably γ1 or γ2), or variants thereof, and one or two linear amino acid probes.

A peptide consisting of for example from 8 to 52 amino acid residues is meant to refer to a peptide amounting in total of from 8 to 52 amino acid residues. This does however not exclude that the peptide is further modified by any other means known to the skilled person, such as being linked to other molecules, being comprised in a larger complex, being post-translationally modified and so forth.

In one embodiment there is provided a peptide consisting of from 9 to 32 amino acids, said peptide comprising the amino acid sequence of γ1-MSH, or variants thereof, said γ1-MSH consisting of from 7 to 12 amino acids, and comprising one linear amino acid probe, said linear amino acid probe consisting of from 2 to 20 amino acids.

In one embodiment there is provided a peptide consisting of from 8 to 31 amino acids, said peptide comprising the amino acid sequence of γ2-MSH, or variants thereof, said γ2-MSH consisting of from 6 to 11 amino acids, and comprising one linear amino acid probe, said linear amino acid probe consisting of from 2 to 20 amino acids.

In one embodiment there is provided a peptide consisting of from 8 to 32 amino acids, said peptide comprising the amino acid sequence of γ-MSH (such as γ1- or γ2-MSH), or variants thereof, said γ-MSH consisting of from 6 to 12 amino acids and comprising one linear amino acid probe, said linear amino acid probe consisting of from 2 to 20 amino acids.

In one embodiment there is provided a peptide consisting of from 11 to 52 amino acids, said peptide comprising the amino acid sequence of γ1-MSH, or variants thereof, said γ1-MSH consisting of from 7 to 12 amino acids, and comprising two linear amino acid probes, each of said linear amino acid probes consisting of from 2 to 20 amino acids.

In one embodiment there is provided a peptide consisting of from 10 to 51 amino acids, said peptide comprising the amino acid sequence of γ2-MSH, or variants thereof, said γ2-MSH consisting of from 6 to 11 amino acids, and comprising two linear amino acid probes, each of said linear amino acid probes consisting of from 2 to 20 amino acids.

In one embodiment there is provided a peptide consisting of from 10 to 52 amino acids, said peptide comprising the amino acid sequence of γ-MSH (such as γ1- or γ2-MSH), or variants thereof, said γ-MSH consisting of from 6 to 12 amino acids and comprising two linear amino acid probes, each of said linear amino acid probes consisting of from 2 to 20 amino acids.

In one embodiment, the present invention is directed to a peptide consisting of from 8 to 52 amino acid residues comprising an amino acid sequence as defined herein above (γ-MSH, or variants thereof, and one or two linear amino acid probes). In a particular embodiment, said peptide consists of from 8 to 9 amino acid residues, for example 9 to 10 amino acid residues, such as 10 to 11 amino acid residues, for example 11 to 12 amino acid residues, such as 12 to 13 amino acid residues, for example 13 to 14 amino acid residues, such as 14 to 15 amino acid residues, for example 15 to 16 amino acid residues, such as from 16 to 17 amino acid residues, for example from 17 to 18 amino acid residues, such as from 18 to 19 amino acid residues, for example from 19 to 20 amino acid residues, such as from 20 to 21 amino acid residues, for example from 21 to 22 amino acid residues, such as from 22 to 23 amino acid residues, for example from 23 to 24 amino acid residues, such as from 24 to 25 amino acid residues, for example from 25 to 26 amino acid residues, such as from 26 to 27 amino acid residues, for example from 27 to 28 amino acid residues, such as from 28 to 29 amino acid residues, for example from 29 to 30 amino acid residues, such as from 30 to 31 amino acid residues, for example from 31 to 32 amino acid residues, such as 32 to 33 amino acid residues, for example 33 to 34 amino acid residues, such as from 34 to 35 amino acid residues, for example from 35 to 36 amino acid residues, such as from 36 to 37 amino acid residues, for example from 37 to 38 amino acid residues, such as from 38 to 39 amino acid residues, for example from 39 to 40 amino acid residues, such as from 40 to 41 amino acid residues, for example from 41 to 42 amino acid residues, such as from 42 to 43 amino acid residues, for example from 43 to 44 amino acid residues, such as from 44 to 45 amino acid residues, for example from 45 to 46 amino acid residues, such as from 46 to 47 amino acid residues, for example from 47 to 48 amino acid residues, such as from 48 to 49 amino acid residues, for example from 49 to 50 amino acid residues, such as from 50 to 51 amino acid residues, for example from 51 to 52 amino acid residues, comprising an amino acid sequence as defined herein above.

### γ-MSH Analogues

It is thus an aspect of the invention to provide a peptide consisting of from 8 to 52 amino acids, said peptide comprising the amino acid sequence:

(aa₁)ₙ-Y-(aa₂)ₘ-Z

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Tip (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe);
wherein said sequence further comprises one or two linear amino acid probes, each consisting of from 2 to 20 consecutive amino acid residues, covalently bound to the N- and/or C-terminus of said amino acid sequence.

It is understood that ‘can be’ may be substitutes with ‘is’ throughout.

In one embodiment there is provided a peptide consisting of from 8 to 52 amino acids, said peptide comprising the amino acid sequence:

{X₁}-(aa₁)ₙ-Y-(aa₂)ₘ-Z-{X₂}

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein {X₁} is an optional linear amino acid probe consisting of from 2 to 20 consecutive amino acid residues covalently linked to (aa₁)ₙ,
wherein {X₂} is an optional linear amino acid probe consisting of from 2 to 20 consecutive amino acid residues covalently linked to Z,
with the proviso that at least {X₁}, or {X₂}, or {X₁} and {X₂}, is present.

In one embodiment said peptide consists of 8 to 52 amino acids, such as 8 to 32, for example 8 to 31, such as 9 to 32, for example 11 to 52, such as 10 to 51, for example 10 to 52.

In one embodiment there is provided a peptide consisting of from 8 to 32 amino acids, said peptide comprising the amino acid sequence:

X₁-(aa₁)ₙ-Y-(aa₂)ₘ-Z

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₁ is a linear amino acid probe consisting of from 2 to 20 consecutive amino acid residues covalently linked to (aa₁)ₙ.

In one embodiment there is provided a peptide consisting of from 10 to 52 amino acids, said peptide comprising the amino acid sequence:

X₁-(aa₁)ₙ-Y-(aa₂)ₘ-Z-X₂

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe);
wherein X₁ is a linear amino acid probe consisting of from 2 to 20 consecutive amino acid residues covalently linked to (aa₁)ₙ, and
X₂ is a linear amino acid probe consisting of from 2 to 20 consecutive amino acid residues covalently linked to Z.

In one embodiment there is provided a peptide consisting of from 8 to 32 amino acids, said peptide comprising the amino acid sequence:

(aa₁)ₙ-Y-(aa₂)ₘ-Z-X₂

wherein n is a number selected from 0, 1, 2, 3 and 4, and (aa₁) independently can be any natural or unnatural amino acid residue;
wherein Y comprises an amino acid sequence selected from the group consisting of His-Phe-Arg-Trp (SEQ ID NO: 1); His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) can be any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₂ is a linear amino acid probe consisting of from 2 to 20 consecutive amino acid residues covalently linked to Z.

In one embodiment there is provided a peptide consisting of a sequence selected from the group consisting of
{X₁}-Tyr-Val-Met-Gly-His-Phe-Arg-Trp-Asp-Arg-Phe-Gly-{X₂} ({X₁}-(SEQ ID NO: 89)-{X₂}),
{X₁}-Tyr-Val-Met-Gly-His-(D-Phe)-Arg-Trp-Asp-Arg-Phe-Gly-{X₂},
{X₁}-Tyr-Val-Met-Gly-His-Phe-(D-Arg)-Trp-Asp-Arg-Phe-Glyv,
{X₁}-Tyr-Val-Met-Gly-His-Phe-Arg-(D-Trp)-Asp-Arg-Phe-Gly-{X₂},
{X₁}-Tyr-Val-Met-Gly-His-(D-Phe)-Arg-(D-Trp)-Asp-Arg-Phe-Gly-{X₂},
{X₁}-Tyr-Val-Met-Gly-His-Nal-Arg-Trp-Asp-Arg-Phe-Gly-{X₂},
{X₁}-Tyr-Val-Met-Gly-His-(D-Nal)-Arg-Trp-Asp-Arg-Phe-Gly-{X₂},
{X₁}-Tyr-Val-Met-Gly-His-Phe-Arg-Trp-Asp-Arg-(D-Phe)-Gly-{X₂},
{X₁}-Tyr-Val-Met-Gly-His-(D-Phe)-Arg-Trp-Asp-Arg-(D-Phe)-Gly-{X₂},
{X₁}-Tyr-Val-Met-Gly-His-Phe-(D-Arg)-Trp-Asp-Arg-(D-Phe)-Gly-{X₂},
{X₁}-Tyr-Val-Met-Gly-His-Phe-Arg-(D-Trp)-Asp-Arg-(D-Phe)-Gly-{X₂},
{X₁}-Tyr-Val-Met-Gly-His-(D-Phe)-Arg-(D-Trp)-Asp-Arg-(D-Phe)-Gly-{X₂},
{X₁}-Tyr-Val-Met-Gly-His-Nal-Arg-Trp-Asp-Arg-(D-Phe)-Gly-{X₂},
{X₁}-Tyr-Val-Met-Gly-His-(D-Nal)-Arg-Trp-Asp-Arg-(D-Phe)-Gly-{X₂},
{X₁}-Tyr-Val-Nle-Gly-His-Phe-Arg-Trp-Asp-Arg-Phe-Gly-{X₂},
{X₁}-Tyr-Val-Nle-Gly-His-(D-Phe)-Arg-Trp-Asp-Arg-Phe-Gly-{X₂},
{X₁}-Tyr-Val-Nle-Gly-His-Phe-(D-Arg)-Trp-Asp-Arg-Phe-Gly-{X₂},
{X₁}-Tyr-Val-Nle-Gly-His-Phe-Arg-(D-Trp)-Asp-Arg-Phe-Gly-{X₂},
{X₁}-Tyr-Val-Nle-Gly-His-(D-Phe)-Arg-(D-Trp)-Asp-Arg-Phe-Gly-{X₂},
{X₁}-Tyr-Val-Nle-Gly-His-Nal-Arg-Trp-Asp-Arg-Phe-Gly-{X₂},
{X₁}-Tyr-Val-Nle-Gly-His-(D-Nal)-Arg-Trp-Asp-Arg-Phe-Gly-{X₂},
{X₁}-Tyr-Val-Nle-Gly-His-Phe-Arg-Trp-Asp-Arg-(D-Phe)-Gly-{X₂},
{X₁}-Tyr-Val-Nle-Gly-His-(D-Phe)-Arg-Trp-Asp-Arg-(D-Phe)-Gly-{X₂},
{X₁}-Tyr-Val-Nle-Gly-His-Phe-(D-Arg)-Trp-Asp-Arg-(D-Phe)-Gly-{X₂},
{X₁}-Tyr-Val-Nle-Gly-His-Phe-Arg-(D-Trp)-Asp-Arg-(D-Phe)-Gly-{X₂},
{X₁}-Tyr-Val-Nle-Gly-His-(D-Phe)-Arg-(D-Trp)-Asp-Arg-(D-Phe)-Gly-{X₂},
{X₁}-Tyr-Val-Nle-Gly-His-Nal-Arg-Trp-Asp-Arg-(D-Phe)-Gly-{X₂}, and
{X₁}-Tyr-Val-Nle-Gly-His-(D-Nal)-Arg-Trp-Asp-Arg-(D-Phe)-Gly-{X₂},
   wherein {X₁} is an optional linear amino acid probe consisting of from 2 to 20 consecutive amino acid residues covalently linked to the most N-terminal Tyr,
   wherein {X₂} is an optional linear amino acid probe consisting of from 2 to 20 consecutive amino acid residues covalently linked to the most C-terminal Gly,
   with the proviso that at least {X₁}, or {X₂}, or {X₁} and {X₂}, is present.

In one embodiment said carboxy terminal Gly is Glycine amide.

In one embodiment there is provided a peptide consisting of a sequence selected from the group consisting of
{X₁}-Tyr-Val-Met-Gly-His-Phe-Arg-Trp-Asp-Arg-Phe-{X₂} ({X₁}-(SEQ ID NO: 90)-{X₂}),
{X₁}-Tyr-Val-Met-Gly-His-(D-Phe)-Arg-Trp-Asp-Arg-Phe-{X₂},
{X₁}-Tyr-Val-Met-Gly-His-Phe-(D-Arg)-Trp-Asp-Arg-Phe-{X₂},
{X₁}-Tyr-Val-Met-Gly-His-Phe-Arg-(D-Trp)-Asp-Arg-Phe-{X₂},
{X₁}-Tyr-Val-Met-Gly-His-(D-Phe)-Arg-(D-Trp)-Asp-Arg-Phe-{X₂},
{X₁}-Tyr-Val-Met-Gly-His-Nal-Arg-Trp-Asp-Arg-Phe-{X₂},
{X₁}-Tyr-Val-Met-Gly-His-(D-Nal)-Arg-Trp-Asp-Arg-Phe-{X₂},
{X₁}-Tyr-Val-Met-Gly-His-Phe-Arg-Trp-Asp-Arg-(D-Phe)-{X₂},
{X₁}-Tyr-Val-Met-Gly-His-(D-Phe)-Arg-Trp-Asp-Arg-(D-Phe)-{X₂},
{X₁}-Tyr-Val-Met-Gly-His-Phe-(D-Arg)-Trp-Asp-Arg-(D-Phe)-{X₂},
{X₁}-Tyr-Val-Met-Gly-His-Phe-Arg-(D-Trp)-Asp-Arg-(D-Phe)-{X₂},
{X₁}-Tyr-Val-Met-Gly-His-(D-Phe)-Arg-(D-Trp)-Asp-Arg-(D-Phe)-{X₂},
{X₁}-Tyr-Val-Met-Gly-His-Nal-Arg-Trp-Asp-Arg-(D-Phe)-{X₂},
{X₁}-Tyr-Val-Met-Gly-His-(D-Nal)-Arg-Trp-Asp-Arg-(D-Phe)-{X₂},
{X₁}-Tyr-Val-Nle-Gly-His-Phe-Arg-Trp-Asp-Arg-Phe-{X₂},
{X₁}-Tyr-Val-Nle-Gly-His-(D-Phe)-Arg-Trp-Asp-Arg-Phe-{X₂},
{X₁}-Tyr-Val-Nle-Gly-His-Phe-(D-Arg)-Trp-Asp-Arg-Phe-{X₂},
{X₁}-Tyr-Val-Nle-Gly-His-Phe-Arg-(D-Trp)-Asp-Arg-Phe-{X₂},
{X₁}-Tyr-Val-Nle-Gly-His-(D-Phe)-Arg-(D-Trp)-Asp-Arg-Phe-{X₂},
{X₁}-Tyr-Val-Nle-Gly-His-Nal-Arg-Trp-Asp-Arg-Phe-{X₂},
{X₁}-Tyr-Val-Nle-Gly-His-(D-Nal)-Arg-Trp-Asp-Arg-Phe-{X₂},
{X₁}-Tyr-Val-Nle-Gly-His-Phe-Arg-Trp-Asp-Arg-(D-Phe)-{X₂},
{X₁}-Tyr-Val-Nle-Gly-His-(D-Phe)-Arg-Trp-Asp-Arg-(D-Phe)-{X₂},
{X₁}-Tyr-Val-Nle-Gly-His-Phe-(D-Arg)-Trp-Asp-Arg-(D-Phe)-{X₂},
{X₁}-Tyr-Val-Nle-Gly-His-Phe-Arg-(D-Trp)-Asp-Arg-(D-Phe)-{X₂},
{X₁}-Tyr-Val-Nle-Gly-His-(D-Phe)-Arg-(D-Trp)-Asp-Arg-(D-Phe)-{X₂},
{X₁}-Tyr-Val-Nle-Gly-His-Nal-Arg-Trp-Asp-Arg-(D-Phe)-{X₂}, and
{X₁}-Tyr-Val-Nle-Gly-His-(D-Nal)-Arg-Trp-Asp-Arg-(D-Phe)-{X₂},
   wherein {X₁} is an optional linear amino acid probe consisting of from 2 to 20 consecutive amino acid residues covalently linked to the most N-terminal Tyr,
   wherein {X₂} is an optional linear amino acid probe consisting of from 2 to 20 consecutive amino acid residues covalently linked to the most C-terminal Phe or (D-Phe),
   with the proviso that at least {X₁}, or {X₂}, or {X₁} and {X₂}, is present.

In one embodiment said carboxy terminal Phe or (D-Phe) is a Phenylalanine amide.

### Embodiments of (aa₁)ₙ

As defined herein above, (aa₁)ₙ of the equation (aa₁)ₙ-Y-(aa₂)ₘ-Z is a sequence consisting of from 0 to 4 amino acids (n=0, 1, 2, 3, or 4). It follows that (aa₁)ₙ may consist of 0 amino acids, or consist of from 1 to 2, such as 2 to 3, for example 3 to 4 contiguous amino acid residues.

In a particular embodiment, (aa₁)ₙ is a sequence consisting of 4 contiguous amino acids (n=4).

In a particular embodiment, (aa₁)ₙ may be an amino acid sequence corresponding to the native part of γ-MSH, or variants thereof. The native part of γ-MSH in this respect is Tyr-Val-Met-Gly.

In one embodiment, (aa₁)ₙ comprises an amino acid sequence selected from the group consisting of Tyr-Val-Met-Gly (SEQ ID NO: 2), Ac-Tyr-Val-Met-Gly, Tyr-Val-Nle-Gly and Ac-Tyr-Val-Nle-Gly. In one embodiment, (aa₁)ₙ comprises an amino acid sequence selected from the group consisting of Tyr-Val-Met-Gly (SEQ ID NO: 2) and Tyr-Val-Nle-Gly. In one embodiment (aa₁)ₙ is Tyr-Val-Met-Gly (SEQ ID NO: 2). In another embodiment, (aa₁)ₙ is Tyr-Val-Nle-Gly.

### Embodiments of Y

In one embodiment, Y is His-Phe-Arg-Trp (SEQ ID NO: 1). In another embodiment, Y is His-(D-Phe)-Arg-Trp. In yet another embodiment, Y is His-Phe-(D-Arg)-Trp. In another embodiment, Y is His-Phe-Arg-(D-Trp). In another embodiment, Y is His-(D-Phe)-Arg-(D-Trp). In another embodiment, Y is His-Nal-Arg-Trp. In another embodiment, Y is His-(D-Nal)-Arg-Trp.

Embodiments of (aa₂)ₘ

As defined herein above, (aa₂)ₘ of the equation (aa₁)ₙ-Y-(aa₂)ₘ-Z is an amino acid residue consisting of 0 or 1 amino acid (m=0 or 1). In one embodiment, (aa₂)ₘ consist of 1 amino acid (m=1).

In one embodiment (aa₂)ₘ is an amino acid corresponding to the native part of γ-MSH, or variants thereof. The native part of γ-MSH in this respect is Asp.

In one embodiment (aa₂)ₘ is Asp (aspartic acid (D)).

In one embodiment (aa₁)ₙ is selected from the group consisting of Tyr-Val-Met-Gly (SEQ ID NO: 2), Ac-Tyr-Val-Met-Gly, Tyr-Val-Nle-Gly and Ac-Tyr-Val-Nle-Gly, and (aa₂)ₘ is Asp.

### Embodiments of Z

In one embodiment, Z is Arg-Phe-Gly. In another embodiment Z is Arg-(D-Phe)-Gly. In yet another embodiment, Z is Arg-Phe. In another embodiment Z is Arg-(D-Phe).

### Activity

The term “agonist” in the present context refers to a substance or a peptide as defined herein, capable of binding to and/or activating a receptor, or in some embodiments, capable of binding to and/or activating a receptor to at least some extent, or in some embodiments, capable of activating a receptor to at least some extent. A MC1r receptor agonist (MC1r agonist) is thus capable of binding to and/or activating the MC1r receptor to at least some extent. The terms ‘MC1r agonist’ and ‘MC1r receptor agonist’ are used interchangeably herein.

An agonist may be an agonist of several different types of receptors, and thus capable of binding and/or activating several different types of receptors. Said agonist can also be a selective agonist which only binds and activates one type of receptor. The term “antagonist” in the present context refers to a substance capable of inhibiting the effect of a receptor agonist.

Full agonists bind (have affinity for) and activate a receptor, displaying full efficacy at that receptor. “Partial agonists” in the present context are peptides able to bind and activate a given receptor, but having only partial efficacy at the receptor relative to a full agonist. Partial agonists can act as antagonists when competing with a full agonist for receptor occupancy and producing a net decrease in the receptor activation compared to the effects or activation observed with the full agonist alone.

“Selective agonists” in the present context are compounds which are selective and therefore predominantly bind and activate one type of receptor. Thus a selective MC1r receptor agonist is selective for the MC1r receptor.

Peptides according to the present invention are in one embodiment capable of binding and activating to some extent one or several melanocortin (MC) receptors and can have different binding affinities and/or different receptor activation efficacy for different MC receptors, wherein affinity refers to the number and size of intermolecular forces between a peptide ligand and its receptor, and residence time of the ligand at its receptor binding site; and receptor activation efficacy refers to the ability of the peptide ligand to produce a biological response upon binding to the target receptor and the quantitative magnitude of this response. In some embodiments, such differences in affinity and receptor activation efficacy are determined by receptor binding/activation studies which are conventional in the art, for instance by generating EC₅₀ and Emax values for stimulation of ligand binding in cells expressing one or several types of MC receptors as mentioned herein, or on tissues expressing the different types of MC receptors. High affinity means that a lower concentration of a compound is needed to obtain a binding of 50% of the receptors compared to peptides which have lower affinity; high receptor activation efficacy means that a lower concentration of the peptide is needed to obtain a 50% receptor activation response (low EC₅₀ value), compared to peptides which have lower affinity and/or receptor activity efficacy (higher EC₅₀ value).

The receptor activation potency of peptide agonists of the present invention can also be measured in p(A₅₀) values which is a conventional method for determining the receptor activation efficacy of an agonist.

In one embodiment of the present invention, the peptides are selective or combined agonists of one or more of the MC receptors selected from MC1r, MC2r, MC3r, MC4r and MC5r.

In one embodiment of the present invention, the peptides are selective agonists of one of the MC receptors selected from MC1r, MC2r, MC3r, MC4r and MC5r.

In one embodiment of the present invention, the peptides are combined agonists of two of the MC receptors selected from MC1r, MC2r, MC3r, MC4r and MC5r.

In one embodiment of the present invention, the peptides are combined agonists of two or more of the MC receptors have differing affinities and/or receptor activation efficacies for two or more of the receptors selected from MC1r, MC2r, MC3r, MC4r and MC5r.

In one particular embodiment, the peptides according to the present invention are capable of binding to and activating at least the melanocortin receptor MC1r. In a further embodiment said peptide is a full agonist of the melanocortin receptor MC1r.

In a further embodiment, said peptide is further capable of binding to and activating melanocortin receptor MC3r. It follows that the peptide of the present invention in one embodiment is capable of binding to and activating the melanocortin receptors MC1r and/or MC3r. In another embodiment, the peptide of the present invention is capable of binding to and activating the melanocortin receptors MC1r and MC3r.

### Methods of Preparation

The peptides according to the present invention may be prepared by any suitable methods known in the art. Thus, in some embodiments the γ-MSH (native or variants as defined herein), and the X motif, are prepared by standard peptide-preparation techniques, such as solution synthesis or solid phase peptide synthesis (SPPS) such as Merrifield-type solid phase synthesis.

The peptides of the invention are in one embodiment prepared by solid phase synthesis by first constructing the pharmacologically active peptide sequence (γ-MSH; native or variants as defined herein), using well-known standard protection, coupling and de-protection procedures, thereafter sequentially coupling the linear amino acid sequence of the motif X onto the active peptide in a manner similar to the construction of the active peptide, and finally cleaving off the entire peptide from the carrier. This strategy yields a peptide, wherein the motif X is covalently bound to the pharmacologically active peptide at the N- or C-terminal nitrogen atom of the peptide.

In one embodiment, the alpha nitrogen on a suitable amino acid in the amino acid sequence is capped with acetyl, using standard acylation techniques, prior to or after coupling of the linear amino acid sequence on the active peptide.

Reactive moieties at the N- and C-termini, which facilitates amino acid coupling during synthesis, as well as reactive side chain functional groups, can interact with free termini or other side chain groups during synthesis and peptide elongation and negatively influence yield and purity. Chemical groups are thus developed that bind to specific amino acid functional groups and block, or protect, the functional group from nonspecific reactions. Purified, individual amino acids are reacted with these protecting groups prior to synthesis and then selectively removed during specific steps of peptide synthesis. Examples of N-terminal protecting groups are t-Boc and Fmoc, commonly used in solid-phase peptide synthesis. C-terminal protecting groups are mostly used in liquid-phase synthesis. Because N-terminal deprotection occurs continuously during peptide synthesis, protecting schemes have been established in which the different types of side chain protecting groups (benzyl; Bzl or tert-butyl; tBu) are matched to either Boc or Fmoc, respectively, for optimized deprotection.

In a particular embodiment of the invention, when preparing the linear amino acid probe, the protection group for Lys is Mtt, which protected amino acid is commercially available (Fmoc-Lys(Mtt)-OH; N-α-Fmoc-N-ε-4-methyltrityl-L-lysine, CAS#167393-62-6). Lys(Mtt) allows for capping Lys with acetyl as it is not cleaved under the conditions that cleave Fmoc, and may be removed without cleavage of other side chain protection groups.

The method of preparation is in some embodiments optimized by routine methods in the art that may increase the yield and/or quality of the thus prepared synthetic peptide. For instance, use of pseudoproline (oxazolidine) dipeptides in the Fmoc SPPS of serine- and threonine-containing peptides may lead to improvements in quality and yield of crude products and may help avoid unnecessary repeat synthesis of failed sequences. These dipeptides are easy to use: simply substitute a serine or threonine residue together with the preceding amino acid residue in the peptide sequence with the appropriate pseudoproline dipeptide. The native sequence is regenerated on cleavage and deprotection.

In one embodiment the sequence of the pharmacologically active peptide sequence (γ-MSH; native or variants as defined herein) and the one or two X-motifs (or parts thereof) are each prepared separately by for example solution synthesis, solid phase synthesis, recombinant techniques, or enzymatic synthesis, followed by coupling of the (at least) two sequences, in some embodiments three sequences, by well-known segment condensation procedures, either in solution or using solid phase techniques, or a combination thereof.

In one embodiment, the γ-MSH as defined herein is prepared by recombinant DNA methods and the X motif is prepared by solid or solution phase synthesis. The conjugation of the γ-MSH and the X motif is in one embodiment carried out by using chemical ligation. This technique allows for the assembling of totally unprotected peptide segments in a highly specific manner. In another embodiment, the conjugation is performed by protease-catalysed peptide bond formation, which offers a highly specific technique to combine totally unprotected peptide segments via a peptide bond.

In one embodiment, the C-terminal amino acid of the X-motif or the C-terminal amino acid of the γ-MSH is attached to the solid support material by means of a common linker such as 2,4-dimethoxy-4′-hydroxy-benzophenone, 4-(4-hydroxy-methyl-3-methoxyphenoxy)-butyric acid, 4-hydroxy-methylbenzoic acid, 4-hydroxymethyl-phenoxyacetic acid, 3-(4-hydroxymethylphenoxyl)propionic acid, or p-{(R,S)-α-[1-(9H-Fluoren-9-yl)-methoxyformamido]-2,4-dimethoxybenzyl}-phenoxyacetic acid (Rink amide linker).

Examples of suitable solid support materials (SSM) are e.g., functionalised resins such as polystyrene, polyacrylamide, polydimethylacrylamide, polyethyleneglycol, cellulose, polyethylene, polyethyleneglycol grafted on polystyrene, latex, dynabeads, etc.

The produced peptides of the invention are in some embodiment cleaved from the solid support material by means of an acid such as trifluoracetic acid, trifluoromethanesulfonic acid, hydrogen bromide, hydrogen chloride, hydrogen fluoride, etc. optionally in combination with one phenol, thioanisole, etc., or the peptide conjugate of the invention are in other embodiments cleaved from the solid support by means of a base such as ammonia, hydrazine, an aikoxide, such as sodium ethoxide, an hydroxide, such as sodium hydroxide, etc.

In other embodiments, the peptides of the invention may be prepared or produced by recombinant techniques. Thus, in one aspect of the present invention the peptide is produced by host cells comprising a first nucleic acid sequence encoding the peptide operably associated with a second nucleic acid capable of directing expression in said host cells. In some embodiments the second nucleic acid sequence comprises or even consists of a promoter that will direct the expression of protein of interest in said cells. A skilled person will be readily capable of identifying useful second nucleic acid sequences (e.g. vectors and plasmids) for use in a given host cell.

The process of producing a recombinant peptide in general comprises the steps of: providing a host cell, preparing a gene expression construct comprising a first nucleic acid encoding the peptide operably linked to a second nucleic acid capable of directing expression of said protein of interest in the host cell, transforming the host cell with the construct and cultivating the host cell, thereby obtaining expression of the peptide. In one embodiment of the invention, the recombinantly produced peptide is excreted by the host cells. The host cell include any suitable host cell known in the art, including prokaryotic cells, yeast cells, insect cells and mammalian cells.

In one embodiment, the recombinant peptide thus produced is isolated by any conventional method and may be linked via conventional peptide bond forming chemistry to any suitably protected linear amino peptide moiety. The skilled person will be able to identify suitable protein isolation steps for purifying the peptide.

### Methods of Treatment

It is an aspect to provide γ-MSH-analogues, or simply peptides, as defined according to the present invention, for use as a medicament.

In another aspect, the present invention provides methods for treatment, prevention or alleviation of an ischemic and/or inflammatory condition in the tissue of one or more organs as mentioned herein. Such methods according to the present invention in one embodiment comprise one or more steps of administration or release of an effective amount of a peptide according to the present invention, or a pharmaceutical composition comprising one or more such peptides, to an individual in need thereof. In one embodiment, such steps of administration or release according to the present invention are simultaneous, sequential or separate.

Ischemia is defined as a reduced/arrested blood flow to one or more organs resulting in a reduced oxygen delivery and/or utilization by the tissues. Ischemia induces multiple tissue reactions including neutrophil accumulation, other inflammatory responses and cell death. Ischemia is involved in multiple diseases, is associated with major surgery, and also occurs secondary to other severe diseases.

An individual in need as referred to herein, is in one embodiment an individual that benefits from the administration of a peptide or pharmaceutical composition according to the present invention. Such an individual in one embodiment suffers from an ischemic and/or inflammatory condition in the tissue of one or more organs, or is at risk of suffering therefrom. The individual is in one embodiment any human being, male or female, infant, middle-aged or old. The disorder to be treated or prevented in the individual in one embodiment relates to the age of the individual, the general health of the individual, the medications used for treating the individual and whether or not the individual has a prior history of suffering from diseases or disorders that may have or have induced ischemic and/or inflammatory conditions in the individual.

The terms “treatment” and “treating” as used herein refer to the management and care of a patient for the purpose of combating a condition, disease or disorder. The term is intended to include the full spectrum of treatments for a given condition from which the patient is suffering, such as administration of the peptide or composition for the purpose of: alleviating or relieving symptoms or complications; delaying the progression of the condition, partially arresting the clinical manifestations, disease or disorder; curing or eliminating the condition, disease or disorder; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The patient to be treated is preferably a mammal, in particular a human being. Treatment of animals, such as mice, rats, dogs, cats, cows, horses, sheep and pigs, is, however, also within the scope of the present invention. The patients to be treated according to the present invention can be of various ages, for example, adults, children, children under 16, children age 6-16, children age 2-16, children age 2 months to 6 years or children age 2 months to 5 years.

The peptides referred to are the γ-MSH-analogues according to the present invention and described in detail herein above.

The invention is in one embodiment directed to a peptide according to the present invention for use in the treatment of an ischemic and/or inflammatory condition in the tissue of one or more organs of a mammal. In one embodiment said treatment is prophylactic, ameliorative and/or curative. In one embodiment, said mammal is a human (homo sapiens).

The invention in certain embodiments is directed to a method for treatment of an ischemic and/or inflammatory condition in the tissue of one or more organs, said method comprising the step of administering a therapeutically effective amount of a peptide according to the present invention to an individual in need thereof.

In a specific embodiment, the invention is directed to use of a peptide according to the present invention for manufacturing of a medicament for the treatment of an ischemic and/or inflammatory condition in the tissue of one or more organs of a mammal.

When referring to the tissue of one or more organs, said organ is in one embodiment selected from the group consisting of kidney, liver, brain, heart, muscles, bone marrow, skin, skeleton, lungs, the respiratory tract, spleen, exocrine glands, bladder, endocrine glands, reproduction organs including the phallopian tubes, eye, ear, vascular system, the gastroinstestinal tract including small intestines, colon, rectum, canalis analis and the prostate gland.

In one embodiment, the ischemic and/or inflammatory condition in the tissue of one or more organs is an acute, subacute or chronic ischemic and/or inflammatory condition.

In one embodiment, the ischemic and/or inflammatory condition in the tissue of one or more organs is an ischemic condition. In another embodiment, the ischemic and/or inflammatory condition in the tissue of one or more organs is an inflammatory condition.

In a further embodiment, the ischemic condition in the tissue of one or more organs is secondary ischemia. Secondary ischemia is ischemia which is caused by an underlying condition such that the ischemia typically is secondary to another condition, such as stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis.

An aortic aneurysm is in one embodiment thoracal or abdominal or dissecting aortic aneurysm. Systemic hypotension is in one embodiment hypotension due to heart disease, hypotension due to systemic disease including infection or allergic reactions, or hypotension due to one or more toxic compound or poison(s) or drug(s).

In one embodiment said ischemic and/or inflammatory condition in the tissue of one or more organs is due to (or caused by) a condition selected from stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis.

In one embodiment, said ischemic condition is myocardial ischemia.

In one embodiment said ischemic and/or inflammatory condition in the tissue of one or more organs is due to cardiac arrhythmia. In one embodiment, said cardiac arrhythmia is the primary disease or secondary to another condition of the individual, including acute infections particularly those affecting the lungs, pulmonary embolism, hypotension, shock, anoxaemia and anaemia.

Cardiac arrhythmias include ventricular or supra ventricular tachyarrhythmias, atrioventricular block, sinus node disease, Wolff-Parkinson—White syndrome, Lenegres disease, Lev's disease any syndrome involving an abnormal myocardial connection between atrium and ventricle.

In one embodiment, secondary ischemia can also be observed in connection with a range of other diseases and conditions, including but not limited to diabetes mellitus, hyperlipidaemia, thromboangiitis obliterans, Takayasu's syndrome, arteritis temporalis, mucocutaneous lymph node syndrome (Kawasaki disease), cardiovascular syphilis, connective tissue disorders such as Raynaud's disease, phlegmasia coerulae dolens, blood vessel trauma including iatrogene trauma such as cannulation, conditions with increased fasting levels of LDL-Cholesterol, triglycerid, and/or HDL—Cholesterol, retroperitoneal fibrosis, rheumatic diseases, systemic lupus erythematosus, polyarteritis nodosa, scleroderma, polymyositis, dermatomyositis, rheumatoid arthritis, neuromyopathic disorders such as progressive muscular dystrophy of Duchenne, Friedreich's ataxia, and myotonic dystrophy, anaphylaxis, serum sickness, hemolytic anaemia, allergy, and allergic agranulocytosis. In one embodiment the peptides of the present invention are also be useful in the treatment or prevention of said conditions.

Many infections may have an influence on the tissue and disturb the normal function resulting in decreased performance, which in one embodiment is treated by administration of an effective dose of a peptide of the invention. In one embodiment, infections include infections by protozoa, virus, bacteria and fungus and include conditions such as AIDS, bacterial septicemia, systemic fungal infections, Rickettsial diseases, toxic shock syndrome, infectious mononucleosis, chlamydia thrachomatis, chlamydia psittaci, cytomegalovirus infection, Campylobacter, salmonella, influenza, poliomyelitis, toxoplasmosis, Lassa Fever, Yellow Fever, billharziose, colibacteria, enterococcus, preteus, klebsiella, pseudomonas, staphylococcus aureus, staphylococcus epidermidis, Candida albicans, tuberculosis, mumps, infectious mononucleosis, hepatitis and Coxackie virus.

In one embodiment the condition to be treated is caused by a cancer or a by premalignant disorder having an impact on the organ, e.g. on the respiratory system including lung, bronchiole, upper airways, and/or on the heart and/or on the kidney and/or on the gastrointestinal system, including acute leukemia, chronic myelocytic leukemia, chronic lymphocytic leukemia, Hodgkin's disease, lymphosarcoma, myeloma, metastasizing carcinoma of any origin. In one embodiment the peptides of the invention are used in the treatment or prevention of said conditions.

In one embodiment, the ischemic and/or inflammatory condition in the tissue of one or more organs is caused by a physical trauma including electromagnetic radiation.

In one embodiment, the inflammatory condition to be treated with a γ-MSH analogue of the invention is a glomerular disease of the kidney, including in one embodiment: diffuse mesangial sclerosis (DMS), congenital nephrotic syndrome of the Finnish type (CNSF), Alport's syndrome and variants (Alport b), MCD, FSGS, collapsing glomerulonephropathy (Collapsing GN), immune and inflammatory glomerulonephropathies (Imm/Inf GN), hypertensive nephropathy (HTN), diabetic glomerulonephropathy (Diab GN), and age-associated glomerulonephropathy (Aging GN) or the induction of diuresis or a remission of proteinuria in the nephrotic syndrome. Together these conditions account for the development of 90% of end-stage kidney disease (ESKD). Glomerulopathies affect the podocytes, which together with the glomerulus basement membranes form filtration slits responsible for allowing water, electrolytes and other small molecules to filter away from blood passing through the nephron and into the urine ducts while retaining proteins (e.g. serum albumin) and cells (e.g., red blood cells and platelets) in the blood. Podocyte injury and death leads to proteinuria (protein in the urine), which represents a health issue in itself, as well as the likelihood of progressing kidney damage.

In treatment of glomuerulopathies, it is known that a deficiency of the melanocortin peptide, ACTH, is associated with nephrotic syndrome caused by FSGS, which inferrs that the melanocortin hormone system might be essential for kidney homeostasis and melanocortin deficiency may predispose to kidney disease. ACTH while primarily binding to the MC2R is also an agoinist at the other melanocortin receptors.

Table I presents MCR 1, 2 and 3 expression in kidney parenchymal cells as well as beneficial effects associated with receptor stimulation by melanocortins (adapted from Gong, R. Advances in Chronic Kidney Disease, 21(2) 133-151 (2014)) as well as blood cells which epress these receptors.

There are several pathways that G-protein coupled receptor (GPCR) MC1-receptor agonists may trigger through several cAMP-PKA pathways, including MAPK/ERK-mediated anti-apoptosis/pro-survival signaling; suppression of NF B-mediated production of inflammatory mediators such as THFα, IL-1β and IL-12; and CREB-mediated stimulation of IL-10 and HO-1 production, both anti-inflammatory and immune tolerance mediators. Another cAMP-PKA mediated pathway that has particular implications for maintenance and remodeling of the foot processes of podocytes are the Rho family proteins (Rho, cdc42 and Rac) that play a role in cytoskeleton remodeling.

Collectively, kidney parenchymal tissues and cells, including podocytes, mesangium, glomerular endothelia, tubular epithelia, and tubulointerstitium, are targets for melanocortin-mediated actions. Accordingly, a γ-MSH analogue according to the present invention may be used to treat diseases or injuries of kidney tissues/cells expressing the MC1 R and MC3 R, including podocytopathies such as MCD (minimal change disease) and FSGS (focal segmental glomerular sclerosis), mesangial diseases such as immunoglobulin A nephropathy (IgAN) and mesangial proliferative glomerulonephritis (MsPGN), glomerular endotheliosis caused by transplant glomerulopathy, preeclampsia, or thrombotic microangiopathy due to hemolytic-uremic syndrome or thrombotic thrombocytopenic purpura, idiopathic thrombocytopenic purpura and tubular injuries such as acute tubular necrosis.

TABLE 1
MCRs in kidney parenchyma

| | MC1 R | MC2 R | MC3R |
|---|---|---|---|
| Agonist | ACTH = α-MSH = β-MSH = γ-MSH | ACTH only | ACTH = α-MSH = β-MSH = γ-MSH |
| preference | | | |
| and affinity | | | |
| Signaling | cAMP pathway | cAMP pathway | cAMP pathway |
| pathways | MAPK/ERK pathway | | IP3-Ca²⁺ pathway |
| Biological | Melanogenesis | Steroidogenesis | Energy homeostasis |
| functions | | | |
| | Antipyresis | | Hemodynamic regulation |
| | Anti-inflammation/ | | |
| | immunomodulation | | |
| | Antiapoptosis [symbol] prosurvival | | |
| | effects | | |
| Expression | Human kidney | Human kidney | Human kidney |
| in the | Mouse kidney | (weak) | Mouse kidney |
| kidney | Rat kidney | | Rat kidney |
| Distribution | Glomeruli | Unknown | Inner medullary collecting |
| in the | Tubular epithelia | | duct |
| kidney | | | |
| Expression | Podocyte | Unknown | Inner medullary |
| in kidney | Mesangial cell | | |
| cells | Glomerular endothelial cell | | |
| | Tubular epithelial cell | | |
| Kidney | Protection of podocytes and | Unknown | Enhanced sodium excretion in |
| effects | other glomerular | cells from | collecting ducts |
| | injury | | |
| | Protection of tubular cells | | |
| | from injury | | |
| | Anti-inflammation | | |
| Clinical | Proteinuria-reducing effects | Unknown | Natriuresis/diuresis |
| effects on | Renoprotection in AKI | | |
| kidney | | | |
| Expression in | Luekocytes: T cells, B cells, NK | Dendritic cells | Macrophages |
| blood cells | cells, APCs | | Monocytes |
| | Monocytes: macrophages, | | Dendritic cells |
| | dendritic cells | | CD4+ cells |
| | | | B leukocytes |

In addition to direct protective effects on kidney cells in glomerular diseases gamma MSH analogs of the present invention can also provide systemic immunomodulation. The analogues of the present invention are able to dampen macrophage mediators in inflammatory settings, by influencing monocytes ability to recruit and prime neutrophils by reducing chemotaxis and inhibiting generation of reactive oxygen species. In another embodiment the gamma MSH anlogues of the invention have significant anti-inflammatory properties mediated through NFkB as demonstrated by α-MSH inhibition of the production or action of proinflammatory factors (nitric oxide, IL-1, IL-6, TNF-α, INFγ, monocyte chemoattractant protein-1), and upregulation of immunosuppressive IL-1β, and downregulation of endothelial adhesion molecules.

Many cells involved in the anti-inflammatory and immunomodulatory actions of melanocortins express MC1R. Melanocortins and their synthetic analogues induce remission of glomerular diseases mainly by targeting the glomerular cells (podocytes, mesangial cells, and glomerular endothelial cells and immune-competence cells via the expressed melanocortin receptors, thereby exerting a direct cellular protective effect on glomerular cells and a systemic immunomodulatory and anti-inflammatory effect.

Suppression of extravastion of leukocytes by down regulation of expression of endothelial adhesion molecules at sites of tissue inflammation as well as immune modulation of antigen presenting cells and production of regulatory T cells which can down regulate production of autoantibodies, both features indicate a beneficial effect in glomerular diseases that are secondary to systemic autoimmune disorders, such as lupus nephritis. In addition the immunomodulation of autoimmune responses and transplant rejection of non-autologous tissues make the alpha and gamma MSH anlogues of the invention useful in treatment of multiple sclerosis, organ transplant rejection and GVHD (graft vs host disease).

In another embodiment, the gamma MSH anlogues of the invention are useful in treating neuroinflammatory pain, such as, for example, neuropathic pain or diabetic neuropathy.

[Abbreviations: adrenocorticotropic hormone; APC, antigen-presenting cells; BC, Bowman's capsule; C, glomerular capillary lumen; E, glomerular endothelial cells; M, mesangium; MCR, melanocortin receptor; MSH, melanocyte-stimulating hormone]

### Surgery and Transplantation

Major surgical interventions including cardiothoracic surgery, abdominal surgery, surgery on the aorta and other major blood vessels, as well as organ transplantation such as lung or heart or combined lung and heart transplantation, liver transplantation or renal transplantation induce a systemic inflammatory response (SIR; or systemic inflammatory response syndrome SIRS) and is associated with post-surgical organ dysfunction including development of renal failure.

Renal failure is a consequence of the SIR and the reduced blood flow generated during the surgical intervention. The result is post-surgical acute kidney injury (AKI) which for a large fraction deteriorates into chronic renal failure. Currently no efficient treatment modality exists to prevent the development of renal failure. Post-surgical renal failure may be defined as a more than 25% reduction in Glomerular filtration rate (GFR) present 3 month after the surgical intervention.

Major cardiac surgery such as repair of one or more cardiac valves, cardiac artery bypass grafting (CABG), surgery on the aortic root, or aortic branch including the common carotic arteries, or combined cardiac surgery such as valve(s) replacement and CABG and/or aortic root surgery is associated with development of renal impairment that, when present, is associated with increased morbidity and mortality.

In one embodiment, treatment with a γMSH analogue according to the present invention reduces the degree of renal impairment. In one embodiment this is achieved by reducing the fall in GFR post-surgery; by reducing the degree of post-surgical increases in serum creatinine or cystatin C or the more immediate increases in urinary excretion of AKI markers NGAL, IL18 or KIM-1; and/or or by reducing the degree of post-surgical SIR (for example by reduced circulating levels of IL-6 and other proiflammatory markers).

Lung transplantation (LTX) is the ultimate treatment modality for end-stage lung disease. The major challenges associated with LTX are scarcity of donors, acute and chronic rejection of the transplanted lungs and side-effects of immune suppressive treatment including development of chronic renal failure (CRF).

While there has been a good development in the treatment of acute rejection by newer immunosuppressive drugs leading to fewer episodes of acute rejection within the first year, fewer organ losses, fewer side effects, fewer infections, and less invasive monitoring methods, the control of chronic organ rejection has not greatly improved and the half-life time in terms of how many years 50% of the patients survive has only marginally improved during the last 2 decades to around 7 years.

Side effects of the immunosuppressive treatment are dominated by 2 major challenges: Nephrotoxicity and post-transplant lymphoproliferative diseases (PTLD), where the latter can be considered as a consequence of the degree of immune-suppression needed to avoid chronic organ rejection—“too much” keeps the rejection on distance, but gives infections and PTLD, while giving “too little” puts the patients at an increased risk of rejecting the graft. Neprotoxicity and development of CRF is, despite extensive research during the last 30 years, still a significant problem. Five years after LTX none of the patients retain normal kidney function and 20% of the long term survivors will end with a kidney transplant as well.

Calcineurin inhibitor treatment (Tacrolimus, Cyclosporin A) is the corner-stone in the immune suppressive treatment strategy for successful solid organ transplantation. The limiting factor in using calcineurin inhibitors is the acute and chronic irreversible nephrotoxicity. Recent data indicate that kidney function (measured as reduction in GFR) is reduced with 40% within the first 14 days after LTX and that this reduction is irreversible.

Heart transplantation (HTX) is the ultimate treatment modality for end-stage heart failure. As for LTX the major challenges associated with HTX are scarcity of donors, acute and chronic rejection of the transplanted hearts and side-effects of immune suppressive treatment including development of CRF. Like for LTX the number of patients with retained kidney function over time is limited or absent and like LTX a major reduction in kidney function is present already two to four weeks post transplantation.

This dramatic effect on kidney function seen after LTX and HTX is probably not caused by calcineurin inhibitor treatment alone, but is the final result of the surgical and anesthesiological trauma in combination with the organ ischemia and side effects of antibiotic, antiviral, antifungal and immunsuppressive drugs. Consequently, in one embodiment pharmacological intervention by employment of the αMSH and γMSH analogues according to the present invention will reduce the degree of renal impairment associated with organ transplantation, such as LTX and HTX.

Surgery, as is outlined herein above in detail, including organ transplantation, may thus be the cause of secondary ischemia.

The invention is thus in one embodiment directed to a peptide according to the present invention for use in the treatment of an ischemic and/or inflammatory condition in the tissue of one or more organs of a mammal, wherein said ischemic and/or inflammatory condition is associated with surgery. In one embodiment said surgery is major surgery or major surgical intervention.

In one embodiment, said surgery is selected from the group consisting of cardiothoracic surgery, abdominal surgery, surgery on the aorta and/or other major blood vessels, repair of one or more cardiac valves, cardiac artery bypass grafting (CABG), surgery on the aortic root or the aortic branch including the common carotic arteries, and combined cardiac surgery such as valve(s) replacement and CABG and/or aortic root surgery.

In one embodiment, said surgery encompasses surgical insertion transplants, devices, grafts, prostheses or other biomedical compounds or devices inserted by surgical operations.

In one embodiment, said major surgery comprises organ transplantation. It follows that the invention in one embodiment is directed to a peptide according to the present invention for use in the treatment of an ischemic and/or inflammatory condition in the tissue of one or more organs of a mammal, wherein said ischemic and/or inflammatory condition is associated with organ transplantation. In one embodiment, said organ transplantation is solid organ transplantation.

In one embodiment said solid organ transplantation is heart transplantation, lung transplantation, combined heart and lung transplantation, liver transplantation or kidney (renal) transplantation.

The invention in another embodiment is directed to a peptide according to the present invention for use in the treatment of post-surgical systemic inflammatory response syndrome (SIRS), post-surgical organ dysfunction and/or post-surgical renal failure such as acute kidney injury (AKI), neprotoxicity and/or chronic renal failure (CRF).

The invention is in one embodiment directed to a peptide according to the present invention for reducing the degree of renal impairment associated with major surgery, in one embodiment, organ transplantation.

Reperfusion injury is tissue damage caused when blood supply returns to the tissue after a period of ischemia or lack of oxygen. The absence of oxygen and nutrients from blood during the ischemic period creates a condition in which the restoration of circulation results in inflammation and oxidative damage through the induction of oxidative stress rather than restoration of normal function.

Reperfusion injuries may occur in connection with surgery, such as major surgical interventions including organ transplantations. It is a primary concern when performing liver transplantations, and also during cardiac surgery.

In a particular embodiment, said ischemic and/or inflammatory condition in the tissue of one or more organs is associated with reperfusion injury. Thus, in one embodiment the present invention is directed to a peptide according to the present invention for use in the treatment of an ischemic and/or inflammatory condition in the tissue of one or more organs of a mammal, wherein said ischemic and/or inflammatory condition is associated with reperfusion injury.

In some embodiments, the peptides or compositions of the present invention are to be administered before and/or during surgery and/or organ transplantation.

### Toxins and Drugs

In one embodiment the ischemic and/or inflammatory condition in the tissue of one or more organs as described herein is caused by toxin- or drug-induced cell, tissue or organ failure.

The invention is thus in one embodiment directed to a peptide according to the present invention for use in the treatment of an ischemic and/or inflammatory condition in the tissue of one or more organs of a mammal, wherein said ischemic and/or inflammatory condition is caused (or induced) by toxin- or drug-induced cell, tissue or organ failure.

Said drug includes but are not restricted to cancer chemotherapeutics including cisplatin, carboplatin, dacarbezine, procarbazine, altretamine, semustine, lomustine, carmustine, busulfan, thiotepa, melphalan, cyclophosphamide, chlorambucil, mechlorethamine, azadtidine, cladrrbine, cytorabine, fludarabine, fluorouracil, mercaptopurine, metrotrexate, thioguanine, allopurinol, bleomycin, dactinomycin, daunorubicin, docetaxel, doxorubicin (adriamycin), etoposide, idarubicin, irinotecan, mitomycin, paclitaxel, plicamycin, topotecan, vinblastine, vincristine, vinorelbine, amasacrine, asparaginase, hydroxyurea, mititane, mitoxantrone; Antibiotics as aminoglycosides including streptomycin, neomycin, kanamycin, amikacin, gentamicin, tobramycin, sisomicin and nitilmicin; immunodepressive compounds as cyclosporine; tricyclic antidepressants, lithium salts, prenylamine and phenothizine derivatives.

### Inflammatory Conditions

Inflammation is a localized defensive response of the body against pathogens and injury. Immune cells and soluble factors take part in this process to neutralize the injurious agent and initiate tissue repair to restore homeostasis. Loss of regulation of these mechanisms can prevent the final resolution of the inflammatory process, leading to chronic inflammation. Chronic inflammation is extremely relevant in today's modern medicine, as it contributes to the pathogenesis of the most important diseases of the industrialized societies including atherosclerosis, acute and chronic heart failure, cancer, diabetes, and obesity-associated diseases. Recent insight into endogenous anti-inflammatory pathways have identified a number of natural anti-inflammatory and pro-resolving molecules and pathways suitable for pharmacological intervention that would make it possible to develop drugs that mimic the natural course of resolving inflammation. Among these natural anti-inflammatory and pro-resolving pathways is the melanocortin system.

The anti-inflammatory effects of melanocortins are at least partly exerted through inhibition of inflammatory mediators and by inhibition of inflammatory cell migration. Melanocortins exert these effects in a variety of cells including monocytes, macrophages, subtypes of T-cells, endothelial cells and epithelial cells.

Most cell types responsive to the anti-inflammatory effect of melanocortins express the MCr1, i.e. monocytes, macrophages, neutrophils, mast cells, fibroblasts, dendritic cells, astrocytes, and microglia. Both human and murine macrophages express the MCr3 and an increasing number of reports have identified MC3r mediated anti-inflammatory effects in vitro and in vivo in models of both acute and more sustained/chronic inflammation.

Consequently, in one embodiment anti-inflammatory intervention targeting the melanocortin system would benefit from targeting both the MC1r and MC3r.

Joint diseases such as rheumatoid arthritis (RA) and gout are characterized by episodes with acute exacerbations, in RA the exacerbations (often described as flairs) typically develop on top of chronic symptoms and develop despite intense pharmacological treatment. A similar pattern can be seen in gout, with the major difference that most gout patients are without symptom between the exacerbations. In both conditions significant neutrophil infiltration into the synovial membrane and joint fluid are the primary pathological hallmark of the exacerbations. The most important proinflammatory effectors involved include IL-1β, TNF-α, IL-6, IL-8, and COX-2. Resolution of the acute exacerbations to avoid development or deterioration of chronic inflammation involves activation of macrophages to phagocytize the apoptotic neutrophils.

Melanocortin type 1 and 3 receptors are expressed in synovial tissue of both animals and humans and it appears that the MC3r is up-regulated in RA patients with active disease.

Consequently in one embodiment treatment is provided with a γMSH analogue according to the present invention to joint diseases, not at least in order to reduce the severity of exacerbations in existing disease as flairs in rheumatoid arthritis would have major clinical impact. However, not only joint diseases are associated with exacerbations of symptoms. Neurodegenerative diseases such as multiple sclerosis have flair-like exacerbations where treatment with a γMSH analogue according to the present invention in one embodiment could reduce the symptoms and eventually as for Joint diseases reduce the overall deterioration of the patients functional level.

The invention is thus in one embodiment directed to a γMSH analogue according to the present invention for use in the treatment of an inflammatory condition in the tissue of one or more organs of a mammal, wherein said ischemic and/or inflammatory condition is an inflammatory disease.

ACTH has been tested in an in vitro cell model of fibrosis by measuring its ability to inhibit TGFβ1-induced differentiation of normal human lung fibroblasts (NHLF) into fibrogenic myofibroblasts producing extracellular matrix (ECM collagen). ACTH demonstrated a statistically significant reduction in ECM collagen at higher doses as detailed in pending U.S. provisional application 61/785,631.

Consequently in one embodiment treatment is provided with a γMSH analogue according to the present invention for use in the treatment of acute respiratory distress syndrome (ARDS) and idiopathic pulmonary fibrosis (IPF).

In one embodiment, said inflammatory disease is arthritis. In one embodiment, said inflammatory disease is selected from the group consisting of an arthropathy (a disease of a joint, arthritis (including diseases associated with arthritis), osteoartritis, rheumatoid arthritis; spondylarthropathies (e.g. ankylosing spondilitis), reactive arthritis (including arthritis following rheumatic fever), Henoch-Schonlein purpura, Reiter's disease, juvenile chronic arthritis including Still's disease, juvenile rheumatoid arthritis, juvenile ankylosing spondylitis, psoriasis, osteoarthritis, osteoarthritis secondary to hypermobilty, congenital dysplasias, slipped femoral epiphysis, Perthes' disease, intra-articular fractures, meniscectomy, obesity, recurrent dislocation, repetitive action injuries, chronic migraine, neuromyelitis optica, crystal depositions and diseases and metabolic abnormalities of cartilage including pyrophosphate arthropathy, ochronosis, haemochromatosis, avascular necrosis including Sickle Cell disease, therapy with corticoids or other drugs, Caisson disease, septic or infectious arthitis (including tuberculous arthritis, meningococcal arthritis, gonococcal arthritis, salmonella arthritis), infective endocarditis, viral arthritis, recurrent haemarthrosis, and all kinds of deposition diseases such as Gout, pyrophosphate arthopathy and acute calcific periarthritis.

In one embodiment, said inflammatory disease is a connective tissue disorder; in one embodiment selected from the group consisting of systemic lupus erythematosus, polymyositis/dermatomyositis, systemic sclerosis, mixed connective tissue disease, sarcoidosis and primary Sjogrens syndrome including keratoconjunctivitis sicca, polymyalgia rheumatica, and other types of vasculitis, crystal deposition diseases (including gout), pyrophosphate arthropathy, and acute calcific periarthritis.

In one embodiment, said inflammatory disease is a soft-tissue rheumatism including bursitis, tenosynovitis or peritendonitis, enthesitis, nerve compression, periarthritis or capsulitis, muscle tension and muscle dysfunction.

In one embodiment, said inflammatory disease is selected from the group consisting of vasculitis including vasculitis secondary to rheumatoid arthritis, infective vasculitis due to infections with bacterial species including spirochaetal diseses as Lyme disease, syphilis, rickettsial and mycobacterial infections, fungal, viral or protozoal infections, non-infective vasculitis secondary to hypersensibility and leucocytoplastic vasculitis including Serum Sickness and Henoch-Schonlein purpura, severe erythema multiforme, Stevens-Johnson syndrome, drug induced vasculitis, essential mixed cryoglobulinaemia, hypocomplentaemia, Vasculitis associated with other kinds of malignancy, non-infective vascultitis including Takayasu's arteritis/disease, giant cell arteritis (temporal arteritis and polymyalgia rheumatica), Buerger's disease, polyarteritis nodosa, microscopic polyarteritis, ANCA-associated vasculitis including Wegener's granulomatosis (also called granulomatosis with polyangitis (GPA)) and Churg-Strauss syndrome (also called eosinophilic granulomatosis with polyanigitis (EGPA)), and vasculitis secondary to connective tissue diseases including Systemic Lupus Erythematosus, Polymyositis/Dermatomyositis, Systemic Sclerosis, Mixed Connective Tissue Disease, sarcoidosis and Primary Sjogrens syndrome.

In one embodiment, said inflammatory disease is inflammatory diseases of the gastrointestinal system. Said inflammatory diseases of the gastrointestinal system may be selected from the group consisting of inflammatory bowel disease, coeliac disease, gluten sensitive enteropathy, eosinophilic gastroenteritis, intestinal lympangiectasia, inflammatory bowel disease (including Crohn's disease and ulcerative colitis), diverticular disease of the colon, radiation enteritis, irritable bowel syndrome, Whipple's disease, stomatitis of all kinds, salivary gland diseases (such as sarcoidosis, salivary duct obstruction and Sjögrens syndrome), inflammation of the oesophagus (e.g. due to gastro-oesophagel reflux or infections with e.g. Candida species, herpes simplex and/or cytomegalus virus), inflammatory diseases of the stomach (including acute and chronic gastritis, helicobacter pylori infection and Mentriers disease), and inflammation of the small intestine.

In one embodiment, said inflammatory disease is a neurodegenerative disease, such as a neurodegenerative disease having an inflammatory component, such as for example multiple sclerosis (MS) or Alzheimer's disease (AD).

In one embodiment, said inflammatory disease is an inflammatory processes involving the eye and its adnexa, including keratitis, iritis, iridocyclitis, diffuse posterior uveitis and choroiditis, optic neuritis, chorioretinitis, and anterior segment inflammation.

In one embodiment, said inflammatory disease is chronic inflammatory demyelinating polyneuropathy (CIDP).

In one embodiment, said inflammatory disease is selected from the group consisting of dermatitis, pemfigus, bulloid pemphigoid, benign mucous membrane pemphigoid, dermatitis herpitiformis, tropical sprue, systemic amyloidosis, primary biliary cirrhosis, Goodpasture syndrome, all kinds of deposition diseases as Gout, pyrophosphate arthopathy and acute calcific periarthritis, pancreatitis, septic discitis, tuberculosis, malignancies (such as matastases, myeloma and others), spinal tumours, ancylosing spondylitis, acute disc prolapse, chronic disc disease/osteoarthritis, osteoporosis, and osteomalacia, Pagets disease, hyperparathyroidism, renal osteodystrophy, spondylolisthesis, spinal senosis congenital abnormalities and fibromyalgia.

In one embodiment, said inflammatory disease is selected from the group consisting of upper and lower airway diseases such as chronic obstructive pulmonary diseases (COPD), allergic and non-allergic asthma, allergic rhinitis, allergic and non-allergic conjunctivitis, allergic and non-allergic dermatitis, acute respiratory distress syndrome (ARDS) and lung inflammation.

### Further Active Ingredients

In some embodiments, the peptides of the present invention are combined with or comprise one or more further active ingredients which in one embodiment are understood as other therapeutical compounds or pharmaceutically acceptable derivatives thereof.

Methods for treatment according to the present invention in one embodiment thus further comprise one or more steps of administration of one or more further active ingredients, either concomitantly or sequentially, and in any suitable ratios. In one embodiment, such further active ingredients is, for example, selected from compounds commonly used to treat or prevent ischemic and/or inflammatory condition in the tissue of one or more organs or symptoms and complications associated with ischemic and/or inflammatory condition in the tissue of one or more organs.

Methods of treatment according to the present invention in one embodiment include a step wherein the pharmaceutical composition or peptide as defined herein is administered simultaneously, sequentially or separately in combination with one or more further active ingredients.

### Administration and Dosage

According to the present invention, a composition comprising a γMSH-analogue as defined herein is in one embodiment administered to individuals in need of treatment in pharmaceutically effective doses or a therapeutically effective amount.

A therapeutically effective amount of a peptide according to the present invention is in one embodiment an amount sufficient to cure, prevent, reduce the risk of, alleviate or partially arrest the clinical manifestations of a given disease or disorder and its complications. The amount that is effective for a particular therapeutic purpose will depend on the severity and the sort of the disorder as well as on the weight and general state of the subject. An amount adequate to accomplish this is defined as a “therapeutically effective amount”.

In one embodiment of the present invention, the peptide or composition is administered in doses of from 1 μg/day to 100 mg/day; such as from 1 μg/day to 10 μg/day, such as 10 μg/day to 100 μg/day, such as 100 μg/day to 250 μg/day, such as 250 μg/day to 500 μg/day, such as 500 μg/day to 750 μg/day, such as 750 μg/day to 1 mg/day, such as 1 mg/day to 2 mg/day, such as 2 mg/day to 5 mg/day, or such as 5 mg/day to 10 mg/day, such as 10 mg/day to 20 mg/day, such as 20 mg/day to 30 mg/day, such as 30 mg/day to 40 mg/day, such as 40 mg/day to 50 mg/day, such as 50 mg/day to 75 mg/day, or such as 75 mg/day to 100 mg/day.

In one embodiment of the present invention, one single dose of the peptide or composition is administered and may comprise of from 1 μg/kg body weight to 100 mg/kg body weight; such as from 1 to 10 μg/kg body weight, such as 10 to 100 μg/day, such as 100 to 250 μg/kg body weight, such as 250 to 500 μg/kg body weight, such as 500 to 750 μg/kg body weight, such as 750 μg/kg body weight to 1 mg/kg body weight, such as 1 mg/kg body weight to 2 mg/kg body weight, such as 2 to 5 mg/kg body weight, such as 5 to 10 mg/kg body weight, such as 10 to 20 mg/kg body weight, such as 20 to 30 mg/kg body weight, such as 30 to 40 mg/kg body weight, such as 40 to 50 mg/kg body weight, such as 50 to 75 mg/kg body weight, or such as 75 to 100 mg/kg body weight.

In one embodiment, a dose according to the present invention is administered one or several times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. In one embodiment, the composition comprising a peptide according to the invention is administered preoperatively (before operation or surgery) and/or peroperatively (during operation or surgery).

### Routes of Administration

It will be appreciated that the preferred route of administration will depend on the general condition and age of the subject to be treated, the nature of the condition to be treated, the location of the tissue to be treated in the body and the active ingredient chosen.

In one embodiment of the present invention, the route of administration allows for the peptide to cross the blood-brain barrier.

### Systemic Treatment

In one embodiment, the route of administration allows for introducing the peptide into the blood stream to ultimately target the sites of desired action.

In one embodiment the routes of administration is any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intravenous and intradermal administration).

Appropriate dosage forms for such administration may be prepared by conventional techniques.

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the peptide or composition is in one embodiment administered topically to cross any mucosal membrane of an animal to which the substance or peptide is to be given, e.g. in the nose, vagina, eye, mouth, genital tract, lungs, gastrointestinal tract, or rectum, for example the mucosa of the nose, or mouth, and accordingly, parenteral administration may also include buccal, sublingual, nasal, rectal, vaginal and intraperitoneal administration as well as pulmonal and bronchial administration by inhalation or installation. In some embodiments, the peptide is administered topically to cross the skin.

In one embodiment, the intravenous, subcutaneous and intramuscular forms of parenteral administration are employed.

### Local Treatment

In one embodiment, the peptide or composition according to the invention is used as a local treatment, i.e. is introduced directly to the site(s) of action. Accordingly, the peptide may be applied to the skin or mucosa directly, or the peptide may be injected into the site of action, for example into the diseased tissue or to an end artery leading directly to the diseased tissue.

### Pharmaceutical Formulations

In one embodiment, the peptides according to the present invention or pharmaceutically acceptable derivatives thereof are administered alone or in combination with pharmaceutically acceptable carriers or excipients, in either single or multiple doses. The pharmaceutical compositions, compounds or peptides according to the invention may be formulated with pharmaceutically acceptable carriers or diluents as well as any other known adjuvants and excipients in accordance with conventional techniques, such as those disclosed in Remington: The Science and Practice of Pharmacy, 20^{th} Edition, Gennaro, Ed., Mack Publishing Co., Easton, Pa., 2000.

The term “pharmaceutically acceptable derivative” in present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes esters and prodrugs, or other precursors of a compound which may be biologically metabolized into the active compound, or crystal forms of a compound.

The pharmaceutical composition or pharmaceutically acceptable composition may be specifically formulated for administration by any suitable route, such as an enteral route, the oral, rectal, nasal, pulmonary, buccal, sublingual, transdermal, intracisternal, intraperitoneal, and parenteral (including subcutaneous, intramuscular, intrathecal, intravenous and intradermal) route.

In an embodiment of the present invention, the pharmaceutical compositions or compounds of the present invention are formulated for crossing the blood-brain-barrier.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art. In the same solid dosage form two active ingredients may be combined so as to provide controlled release of one active ingredient and immediate release of another active ingredient.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also regarded as being within the scope of the present invention.

Other suitable administration forms include suppositories, sprays, ointments, cremes/lotions, gels, inhalants, dermal patches, implants, etc.

In one embodiment, a compound or peptide according to the present invention is generally utilized as the free substance or as a pharmaceutically derivative such as a pharmaceutically acceptable ester or such as a salt thereof. Examples of the latter are: an acid addition salt of a compound having a free base functionality, and a base addition salt of a compound having a free acid functionality. The term “pharmaceutically acceptable salt” refers to a non-toxic salt of a compound for use according to the present invention, which salts are generally prepared by reacting a free base with a suitable organic or inorganic acid, or by reacting an acid with a suitable organic or inorganic base. When a compound according to the present invention contains a free base functionality, such salts are prepared in a conventional manner by treating a solution or suspension of the compound with a chemical equivalent of a pharmaceutically acceptable acid. When a compound according to the present invention contains a free acid functionality, such salts are prepared in a conventional manner by treating a solution or suspension of the compound with a chemical equivalent of a pharmaceutically acceptable base. Physiologically acceptable salts of a compound with a hydroxy group include the anionic form of the compound in combination with a suitable cation, such as sodium or ammonium ion. Other salts which are not pharmaceutically acceptable may be useful in the preparation of compounds of the invention, and these form a further aspect of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, trifluoroacetate, trichloroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In one embodiment, the peptides of the present invention are on crystalline forms, for example co-crystallized forms or hydrates of crystalline forms.

The term “prodrug” refers to peptides that are rapidly transformed in vivo to yield the parent compound of the above formulae, for example, by hydrolysis in blood or by metabolism in cells, such as for example the cells of the basal ganglia. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include pharmaceutically acceptable, non-toxic esters of the compounds of the present invention. Esters of the compounds of the present invention may be prepared according to conventional methods “March's Advanced Organic Chemistry, 5^{th} Edition”. M. B. Smith & J. March, John Wiley & Sons, 2001.

In one embodiment, for parenteral administration, solutions of peptides according to the present invention in sterile aqueous solution, in aqueous propylene glycol or in sesame or peanut oil are employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the compounds according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units, such as capsules or tablets, which each contain a predetermined amount of the active ingredient, and which may include a suitable excipient.

Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, the contents of which are incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the compound for use according to the present invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising peptides for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the compound with a suitable non-irritating excipient which is solid at ordinary temperatures but liquid at the rectal temperature and will thus melt in the rectum to release the drug. Such materials include, for example, cocoa butter and polyethylene glycols.

Peptides of the present invention may also be administered in the form of liposome delivery systems, such as small unilamellar vesicles, large unilamellar vesicles, and multilamellar vesicles. Liposomes may be formed from a variety of phospholipids, such as but not limited to cholesterol, stearylamine or phosphatidylcholines.

In addition, some peptides of the present invention may form solvates with water or common organic solvents. Such solvates are also encompassed within the scope of the invention.

Thus, a further embodiment provides a pharmaceutical composition comprising a peptide for use according to the present invention, or a pharmaceutically acceptable salt, solvate, or prodrug thereof, and one or more pharmaceutically acceptable carriers, excipients, or diluents.

### EXAMPLES

The potency and efficacy of the presently claimed invention can be determined using different pharmacological procedures. The present invention is further illustrated with reference to the following examples, which are not intended to be limiting in any way to the scope of the invention as claimed.

In the following methods for testing the peptides of the invention are described in general. The aim of the methods is to test the peptides of the invention for receptor binding affinity and the efficacy against MC1r, MC2r, MC3r, MC4r and/or MC5r (for instance human or murine).

The immune modulating effects of melanocortins are mediated through MC1r and/or MC3r stimulation on immune competent cells in tissues, organs and plasma. MC1r and/or MC3r are expressed in immune competent cells including monocytes, macrophages, neutrophils t-cells and dendritic cells. Stimulation of the MCr1 and/or MC3r is associated with attenuation of cytokine production and activation of pro-resolving effects. The binding affinity and the receptor efficacy (alternative expression is potency) of a given melanocortin together, in some embodiments, makes up the overall efficacy of a given compound. The degree of a given compounds' binding affinity against the MCr's is defined as the ability to displacement of a radio-labelled full agonist with high binding affinity to the receptor, in the given case displacement of ¹²⁵I-NDP-αMSH from the MCr1. The binding affinity is expressed with an inhibition constant IC₅₀ defined as the concentration of a given compound inducing 50% displacement of the radio-labelled compounds (the lower IC₅₀ the higher binding affinity). The receptor efficacy is defined as the ability to stimulate cAMP production compared to a full agonist as αMSH or NDP-αMSH. Both with regard to maximal efficacy (Eₘₐₓ) and with regard the efficacy constant EC₅₀, defined as the concentration of agonist given ½ max response (the lower EC₅₀ the higher efficacy).

### Experimental Set-Up

Test 1) Binding Affinity Against the Human MC1r, MC3r, MC4r and/or MC5r

Binding affinity against the human MC1r, MC3r, MC4r and/or MC5r is tested using a radioligand binding assay with membrane fraction of CRO cells stably expressing the human MC1r, MC3r, MC4r and/or MC5r.

Competition binding is performed in the wells of a 96 well plate (Master Block, Greiner, 786201) containing binding buffer, humane MC1r membrane extracts, [125I](Lys11)(Nle4-D-Phe7)-a-MSH and test compound at increasing concentrations. The samples are incubated in a final volume of 0.1 ml for 60 min at 25° C. and then filtered over filters. Filters are washed six times with 0.5 ml of ice-cold washing buffer and 50 μl of Microscint 20 (Packard) is added in each well. The plates are incubated 15 min on an orbital shaker and then counted with a TopCount gamma counter for 1 min/well.

Data is presented as mean values. The inhibition constant is determined by best fit analyses after logarithmic transformation using the graph pad software (version 6.0). Differences are considered significant at probability levels (p) of 0.05.

Test 2) Receptor Efficacy Against the Human MC1r, MC3r, MC4r and/or MC5r

CHO-K1 cells expressing either the MC1r, the MC3r, the MC4r or the MC5r grown in media without antibiotic are detached by gentle flushing with PBS-EDTA (5 mM EDTA), recovered by centrifugation and resuspended in assay buffer (KRH: 5 mM KCl, 1.25 mM MgSO4, 124 mM NaCl, 25 mM HEPES, 13.3 mM Glucose, 1.25 mM KH2PO4, 1.45 mM CaCl2, 0.5 g/l BSA).

12 μl of cells are mixed with 12 μl of the test compound at increasing concentrations in 96 wells plates and then incubated 30 min at room temperature. cAMP production is determined after addition of a lysis buffer and 1 hour incubation, by use of competitive immunoassay using cryptate-labeled anti-cAMP and d2-labeled cAMP (HTRF kit from CisBio) with Delta F percentage values calculated according to the manufacturer specification. Dose response curves are performed in parallel with test compounds, and reference compounds.

The HTRF technology is a titration assay based on a competition between labeled cAMP (exogenous) and cAMP produced by the cell after activation of the MCr. The dynamic range of the assay is 3-4 fold meaning that the linear range (which enables conversion from raw data to nM of cAMP) is within that range. The window between top and bottom of the curve is higher (around 100) which means that converting into nM of cAMP, the assay window of cAMP goes from 1 nM (basal) to around 30 nM (Emax). All experiments are conducted in the presence of the non-selective phosphodiesterase inhibitor IBMX (1 mM in final concentration).

Data is presented as mean values. The inhibition constant is determined by best fit analyses after logarithmic transformation using the graph pad software (version 6.0). Differences are considered significant at probability levels (p) of 0.05.

### Example 1

The following γ-MSH analogues are synthesized:

### (Lys₆)-Tyr-Val-Met-Gly-His-Phe-Arg-(D-Trp)-Asp-Arg-Phe-Gly-NH₂

### Tyr-Val-Met-Gly-His-Phe-Arg-(D-Trp)-Asp-Arg-Phe-Gly-(Lys₆)

### (Lys₆)-Tyr-Val-Met-Gly-His-Phe-Arg-(D-Trp)-Asp-Arg-Phe-Gly-(Lys₆)

### (Glu₄)-Tyr-Val-Met-Gly-His-Phe-Arg-Trp-Asp-Arg-Phe-Gly-NH₂

### (Glu₆)-Tyr-Val-Met-Gly-His-Phe-Arg-(D-Trp)-Asp-Arg-Phe-Gly-NH₂

### (Lys₆)-Tyr-Val-Met-Gly-His-Phe-Arg-(D-Trp)-Asp-Arg-Phe-Gly-(Glu₆)

### (Lys-Glu-Lys-Glu-Lys-Glu)-Tyr-Val-Met-Gly-His-Phe-Arg-(D-Trp)-Asp-Arg-Phe-Gly-(Lys-Glu-Lys-Glu-Lys-Glu)

The γ-MSH analogues are tested as outlined above; test 1) binding affinity against the human MC1r, MC2r, MC3r, MC4r and/or MC5r, test 2) receptor efficacy against the human MC1r, MC2r, MC3r, MC4r and/or MC5r, and/or test 3) receptor efficacy against the human MC1r.

### Example 2

### Synthesis of γ-MSH Analogues

Peptides are manufactured using Fmoc (9-fluorenylmethyloxycarbonyl) chemistry. Peptides are made using a polystyrene resin, functionalized with an appropriate linker, and the peptides are then manufactured using an Intavis Peptide Synthesizer. A 4-fold excess of amino acid is added relative to the resin and either HATU (O-(7-azabenzotriazol-1-yl)-N,N,N′,N′-tetramethyluronium hexafluorophosphate) or HCTU (2-(6-Chloro-1H-benzotriazole-1-yl)-1,1,3,3-tetramethylaminium hexafluorophosphate) is used at a 3.95-fold excess to create the active ester. Along with an 8-fold excess of DIPEA (N,N-Diisopropylethylamine) as the base, these reagents catalyze the addition of the next amino acid. Once the amino acid is coupled (each cycle includes a double coupling cycle to insure efficient coupling) the resin is exposed to 20% acetic anhydride to terminate (“cap-off”) any peptide chains that have not added the next amino acid.

The amino acids are dissolved in NMP (N-Methyl-2-pyrrolidone) or DMF (Dimethylformamide) for washing. Piperidine is used to remove the Fmoc group at the end of each coupling cycle which allows the next amino acid to be added.

For the γ-MSH analogue the addition of one or more pseudoproline (oxazolidine) dipeptides during the synthesis of serine- and/or threonine-containing peptides results in improvements in peptide quality and an increase in the yield of full length crude peptide. The peptide is made up to the MEHF, a pseudoproline dipeptide (Fmoc-YS) was added, the next amino acid “Ser” was coupled 3 times to insure it went to completion, and the peptide finished manually by adding the Lys(Mtt), acetylating, and then finishing as above.

The peptides are dried using MeOH (3×), DCM (3×), cleaved using 92% TFA, 2% water, 2% triisopropylsilane, 2% thioanisole and 2% ethanedithiol for 3-4 h at room temperature. Peptides are precipitated in cold diethyl ether, centrifuged (2,000 RPM) and the pellets washed 2× with cold ether. After drying the peptides are solubilized in water containing 0.1% TFA (buffer A) and subjected to RP-HPLC using C18 columns (buffer B=95% acetonitrile/0.1% TFA).

The purity is determined by analytical HPLC and theoretical mono isotopic molecular masses are confirmed by MS. The sequence integrity is verified by CID tandem MS/MS sequencing.

### Example 3

### Synthesis of γ-MSH Analogues

Peptides are manufactured using Fmoc (9-fluorenylmethyloxycarbonyl) chemistry adding individual amino acids sequentially in the desired sequence of the peptide chain.

Peptides are made using a polystyrene resin, functionalized with an appropriate linker, and the peptides are then manufactured using an Intavis Peptide Synthesizer. A 4-fold excess of amino acid is added relative to the resin and either HATU (O-(7-azabenzotriazol-1-yl)-N,N,N′,N′-tetramethyluronium hexafluorophosphate) or HCTU (2-(6-Chloro-1H-benzotriazole-1-yl)-1,1,3,3-tetramethylaminium hexafluorophosphate) is used at a 3.95-fold excess to create the active ester. Along with an 8-fold excess of DIPEA (N,N-Diisopropylethylamine) as the base, these reagents catalyze the addition of the next amino acid in the sequence. Once the amino acid is coupled (each cycle includes a doublecoupling cycle to insure efficient coupling) the resin is exposed to 20% acetic anhydride to terminate (“cap-off”) any peptide chains that have not added the next amino acid.

Piperidine is used to remove the Fmoc group at the end of each coupling cycle which allows the next amino acid to be added. Washing in between the coupling, capping and deprotecting steps is done by suspending the resin in NMP (N-Methyl-2-pyrrolidone) or DMF (Dimethylformamide). The peptides are acetylated at the N-terminal after removal of the Fmoc group, followed by exposure to 20% acetic anhydride.

The resin-linked peptides are subsequently dried using MeOH (3×), DCM (3×), cleaved from the resin and deprotected on the sidechains using 92% TFA, 2% water, 2% triisopropylsilane, 2% thioanisole and 2% ethanedithiol for 3-4 h at room temperature. Peptides are precipitated in cold diethyl ether, centrifuged (2,000 RPM) and the pellets washed 2× with cold ether. After drying the peptides are solubilized in water containing 0.1% TFA (buffer A) and subjected to RP-HPLC using C18 columns (buffer B=95% acetonitrile/0.1% TFA).

The purity is determined by analytical HPLC and theoretical mono isotopic molecular masses are confirmed by MS. The sequence integrity is verified by CID tandem MS/MS sequencing.

What is claimed is:

1. A peptide consisting of from 8 to 52 amino acids, said peptide comprising the amino acid sequence:
{X₁}-(aa₁)ₙ-Y-(aa₂)ₘ-Z-{X₂}
wherein n is a number selected from 0, 1, 2, 3 and 4, and (aa₁) independently is any natural or unnatural amino acid residue;
wherein Y comprises an amino acid sequence selected from the group consisting of His-Phe-Arg-Trp; His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) is any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein {X₁} is an optional linear amino acid probe consisting of from 2 to 20 consecutive amino acid residues covalently linked to (aa₁)ₙ,
wherein {X₂} is an optional linear amino acid probe consisting of from 2 to 20 consecutive amino acid residues covalently linked to Z,
with the proviso that {X₁}, or {X₂}, or {X₁} and {X₂}, is present.

2. The peptide according to claim 1, wherein said peptide consists of from 8 to 32 amino acids and comprises the amino acid sequence
X₁-(aa₁)ₙ-Y-(aa₂)ₘ-Z
wherein n is a number selected from 0, 1, 2, 3 and 4, and (aa₁) independently is any natural or unnatural amino acid residue;
wherein Y comprises an amino acid sequence selected from the group consisting of His-Phe-Arg-Trp; His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) is any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₁ is a linear amino acid probe consisting of from 2 to 20 consecutive amino acid residues covalently linked to (aa₁)ₙ.

3. The peptide according to claim 1, wherein said peptide consists of from 8 to 32 amino acids and comprises the amino acid sequence
(aa₁)ₙ-Y-(aa₂)ₘ-Z-X₂
wherein n is a number selected from 0, 1, 2, 3 and 4, and (aa₁) independently is any natural or unnatural amino acid residue;
wherein Y comprises an amino acid sequence selected from the group consisting of His-Phe-Arg-Trp; His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) is any natural or unnatural amino acid residue;
wherein Z comprises an amino acid sequence selected from the group consisting of Arg-Phe-Gly; Arg-(D-Phe)-Gly; Arg-Phe and Arg-(D-Phe); and
wherein X₂ is a linear amino acid probe consisting of from 2 to 20 consecutive amino acid residues covalently linked to Z

4. The peptide according to claim 1, wherein said peptide consists of from 10 to 52 amino acids, said peptide comprising the amino acid sequence:
X₁-(aa₁)ₙ-Y-(aa₂)ₘ-Z-X₂
wherein n is a number selected from 0, 1, 2, 3 and 4, and (aa₁) independently is any natural or unnatural amino acid residue;
wherein Y comprises an amino acid sequence selected from the group consisting of His-Phe-Arg-Trp; His-(D-Phe)-Arg-Trp; His-Phe-(D-Arg)-Trp; His-Phe-Arg-(D-Trp); His-(D-Phe)-Arg-(D-Trp); His-Nal-Arg-Trp and His-(D-Nal)-Arg-Trp;
wherein m is 0 or 1, and (aa₂) is any natural or unnatural amino acid residue;
wherein Z comprises an amino acid sequence selected from the group consisting of Arg-Phe-Gly; Arg-(D-Phe)-Gly; Arg-Phe and Arg-(D-Phe);
wherein X₁ is a linear amino acid probe consisting of from 2 to 20 consecutive amino acid residues covalently linked to (aa₁)ₙ; and
wherein X₂ is a linear amino acid probe consisting of from 2 to 20 consecutive amino acid residues covalently linked to Z.

5. The peptide according to claim 1, wherein (aa₁)ₙ is a sequence consisting of 4 contiguous amino acids (n=4).

6. The peptide according to claim 1, wherein (aa₁)ₙ comprises an amino acid sequence selected from the group consisting of Tyr-Val-Met-Gly and Tyr-Val-Nle-Gly, and wherein (aa₂)ₘ is Asp.

7. The peptide according to claim 1, wherein Y is selected from the group consisting of His-Phe-Arg-Trp, His-(D-Phe)-Arg-Trp, His-Phe-(D-Arg)-Trp, His-Phe-Arg-(D-Trp), His-(D-Phe)-Arg-(D-Trp), His-Nal-Arg-Trp, and His-(D-Nal)-Arg-Trp.

8. The peptide according to claim 1, wherein (aa₂)ₘ is 1 amino acid (m=1).

9. The peptide according to claim 1, wherein Z is selected from the group consisting of Arg-Phe-Gly, Arg-(D-Phe)-Gly, Arg-Phe, and Arg-(D-Phe).

10. The peptide according to claim 1, wherein said one or two linear amino acid probes individually consist of 2 to 20 consecutive amino acid residues, for example 2 to 3, such as 3 to 4, for example 4 to 5, such as 5 to 6, for example 6 to 7, such as 7 to 8, for example 8 to 9, such as 9 to 10, for example 10 to 11, such as 11 to 12, for example 12 to 13, such as 13 to 14, for example 14 to 15, such as 15 to 16, for example 16 to 17, such as 17 to 18, for example 18 to 19, such as 19 to 20 consecutive amino acid residues.

11. The peptide according to claim 1, wherein said amino acid residues of said one or two linear amino acid probes are individually selected from the group consisting of Ala, Arg, Asn, Asp, Cys, Gln, Glu, Gly, His, Ile, Leu, Lys, Met, Phe, Pro, Ser, Tyr, Thr, Trp, Val, Sec and Pyl.

12. The peptide according to claim 1, wherein said amino acid residues of said one or two linear amino acid probes are individually in the L- or the D-configuration.

13. The peptide according to claim 1, wherein one, two, three, four, five, six, seven, eight, nine or ten of the amino acids of the linear amino acid probe are individually selected from the group consisting of Lys, (D-Lys), Glu and (D-Glu).

14. The peptide according to claim 1, wherein said amino acid residues of said one or two linear amino acid probes are individually selected from the group consisting of Lys, (D-Lys), Glu and (D-Glu).

15. The peptide according to claim 1, wherein each of said amino acid residues of said one or two linear amino acid probes is Lys or (D-Lys).

16. The peptide according to claim 1, wherein said linear amino acid probe is selected from the group consisting of Lys-Lys (Lys₂); Lys-Lys-Lys (Lys₃); Lys-Lys-Lys-Lys (Lys₄); Lys-Lys-Lys-Lys-Lys (Lys₅); Lys-Lys-Lys-Lys-Lys-Lys (Lys₆); Lys-Lys-Lys-Lys-Lys-Lys-Lys (Lys₇); Lys-Lys-Lys-Lys-Lys-Lys-Lys-Lys (Lys₈); Lys-Lys-Lys-Lys-Lys-Lys-Lys-Lys-Lys (Lys₉); Lys-Lys-Lys-Lys-Lys-Lys-Lys-Lys-Lys-Lys (Lys₁₀); Lys-Lys-Lys-Lys-Lys-Lys-Lys-Lys-Lys-Lys-Lys (Lys₁₁); Lys-Lys-Lys-Lys-Lys-Lys-Lys-Lys-Lys-Lys-Lys-Lys (Lys₁₂); Lys-Lys-Lys-Lys-Lys-Lys-Lys-Lys-Lys-Lys-Lys-Lys-Lys (Lys₁₃); Lys-Lys-Lys-Lys-Lys-Lys-Lys-Lys-Lys-Lys-Lys-Lys-Lys-Lys (Lys₁₄) and Lys-Lys-Lys-Lys-Lys-Lys-Lys-Lys-Lys-Lys-Lys-Lys-Lys-Lys-Lys (Lys₁₅), wherein each Lys may individually be selected from Lys and (D-Lys).

17. The peptide according to claim 1, wherein said linear amino acid probe is selected from the group consisting of Glu-Glu (Glu₂); Glu-Glu-Glu (Glu₃); Glu-Glu-Glu-Glu (Glu₄); Glu-Glu-Glu-Glu-Glu (Glu₅); Glu-Glu-Glu-Glu-Glu-Glu (Glu₆); Glu-Glu-Glu-Glu-Glu-Glu-Glu (Glu₇); Glu-Glu-Glu-Glu-Glu-Glu-Glu-Glu (Glu₈); Glu-Glu-Glu-Glu-Glu-Glu-Glu-Glu-Glu (Glu₉); Glu-Glu-Glu-Glu-Glu-Glu-Glu-Glu-Glu-Glu (Glu₁₀); Glu-Glu-Glu-Glu-Glu-Glu-Glu-Glu-Glu-Glu-Glu (Glu₁₁); Glu-Glu-Glu-Glu-Glu-Glu-Glu-Glu-Glu-Glu-Glu-Glu (Glu₁₂); Glu-Glu-Glu-Glu-Glu-Glu-Glu-Glu-Glu-Glu-Glu-Glu-Glu (Glu₁₃); Glu-Glu-Glu-Glu-Glu-Glu-Glu-Glu-Glu-Glu-Glu-Glu-Glu-Glu (Glu₁₄) and Glu-Glu-Glu-Glu-Glu-Glu-Glu-Glu-Glu-Glu-Glu-Glu-Glu-Glu-Glu (Glu₁₅), wherein each Glu may individually be selected from Glu and (D-Glu).

18. The peptide according to claim 1, wherein said linear amino acid probe is selected from the group consisting of Glu-Lys-Lys-Lys-Lys-Lys, Lys-Glu-Lys-Lys-Lys-Lys, Lys-Lys-Glu-Lys-Lys-Lys, Lys-Lys-Lys-Glu-Lys-Lys, Lys-Lys-Lys-Lys-Glu-Lys, Lys-Lys-Lys-Lys-Lys-Glu, Glu-Glu-Lys-Lys-Lys-Lys, Glu-Lys-Glu-Lys-Lys-Lys, Glu-Lys-Lys-Glu-Lys-Lys, Glu-Lys-Lys-Lys-Glu-Lys, Glu-Lys-Lys-Lys-Lys-Glu, Lys-Glu-Glu-Lys-Lys-Lys, Lys-Glu-Lys-Glu-Lys-Lys, Lys-Glu-Lys-Lys-Glu-Lys, Lys-Glu-Lys-Lys-Lys-Glu, Lys-Lys-Glu-Glu-Lys-Lys, Lys-Lys-Glu-Lys-Glu-Lys, Lys-Lys-Glu-Lys-Lys-Glu, Lys-Lys-Lys-Glu-Glu-Lys, Lys-Lys-Lys-Glu-Lys-Glu, Lys-Lys-Lys-Lys-Glu-Glu, Glu-Glu-Glu-Lys-Lys-Lys, Glu-Glu-Lys-Glu-Lys-Lys, Glu-Glu-Lys-Lys-Glu-Lys, Glu-Glu-Lys-Lys-Lys-Glu, Glu-Lys-Glu-Glu-Lys-Lys, Glu-Lys-Glu-Lys-Glu-Lys, Glu-Lys-Glu-Lys-Lys-Glu, Glu-Lys-Lys-Glu-Glu-Lys, Glu-Lys-Lys-Glu-Lys-Glu, Glu-Lys-Lys-Lys-Glu-Glu, Lys-Lys-Lys-Glu-Glu-Glu, Lys-Lys-Glu-Lys-Glu-Glu, Lys-Lys-Glu-Glu-Lys-Glu, Lys-Lys-Glu-Glu-Glu-Lys, Lys-Glu-Lys-Lys-Glu-Glu, Lys-Glu-Lys-Glu-Lys-Glu, Lys-Glu-Lys-Glu-Glu-Lys, Lys-Glu-Glu-Lys-Lys-Glu, Lys-Glu-Glu-Lys-Glu-Lys, Lys-Glu-Glu-Glu-Lys-Lys, Lys-Lys-Glu-Glu-Glu-Glu, Lys-Glu-Lys-Glu-Glu-Glu, Lys-Glu-Glu-Lys-Glu-Glu, Lys-Glu-Glu-Glu-Lys-Glu, Lys-Glu-Glu-Glu-Glu-Lys, Glu-Lys-Lys-Glu-Glu-Glu, Glu-Lys-Glu-Lys-Glu-Glu, Glu-Lys-Glu-Glu-Lys-Glu, Glu-Lys-Glu-Glu-Glu-Lys, Glu-Glu-Lys-Lys-Glu-Glu, Glu-Glu-Lys-Glu-Lys-Glu, Glu-Glu-Lys-Glu-Glu-Lys, Glu-Glu-Glu-Lys-Lys-Glu, Glu-Glu-Glu-Lys-Glu-Lys, Glu-Glu-Glu-Glu-Lys-Lys, Lys-Glu-Glu-Glu-Glu-Glu, Glu-Lys-Glu-Glu-Glu-Glu, Glu-Glu-Lys-Glu-Glu-Glu, Glu-Glu-Glu-Lys-Glu-Glu, Glu-Glu-Glu-Glu-Lys-Glu, Glu-Glu-Glu-Glu-Glu-Lys, Glu-Glu-Glu-Glu-Glu-Glu; wherein each Lys may individually be selected from Lys and (D-Lys) and wherein each Glu may individually be selected from Glu and (D-Glu).

19. The peptide according to claim 1 consisting in total of 14 to 52 amino acids, such as 15 to 32, for example 14 to 31, such as 14 to 32, for example 18 to 52, such as 17 to 51, for example 17 to 52 amino acid residues.

20. The peptide according to claim 1, wherein the amino terminus of said peptide is (B4)HN—, wherein B4 is B4-C(═O)— and B4 is CH₃.

21. The peptide according to claim 1, wherein the amino terminus of said peptide is (B6)HN—, wherein B6 is H.

22. The peptide according to claim 1, wherein the carboxy terminus of said peptide is —C(═O)—B1, wherein B1 is OH or NH₂.

23. The peptide according to claim 1, wherein the carboxy terminus of said peptide is modified by amidation (—NH₂).

24. The peptide according to claim 1, wherein said peptide consists of a sequence selected from the group consisting of
{X₁}-Tyr-Val-Met-Gly-His-Phe-Arg-Trp-Asp-Arg-Phe-Gly-{X₂},
{X₁}-Tyr-Val-Met-Gly-His-(D-Phe)-Arg-Trp-Asp-Arg-Phe-Gly-{X₂},
{X₁}-Tyr-Val-Met-Gly-His-Phe-(D-Arg)-Trp-Asp-Arg-Phe-Gly-{X₂},
{X₁}-Tyr-Val-Met-Gly-His-Phe-Arg-(D-Trp)-Asp-Arg-Phe-Gly-{X₂},
{X₁}-Tyr-Val-Met-Gly-His-(D-Phe)-Arg-(D-Trp)-Asp-Arg-Phe-Gly-{X₂},
{X₁}-Tyr-Val-Met-Gly-His-Nal-Arg-Trp-Asp-Arg-Phe-Gly-{X₂},
{X₁}-Tyr-Val-Met-Gly-His-(D-Nal)-Arg-Trp-Asp-Arg-Phe-Gly-{X₂},
{X₁}-Tyr-Val-Met-Gly-His-Phe-Arg-Trp-Asp-Arg-(D-Phe)-Gly-{X₂},
{X₁}-Tyr-Val-Met-Gly-His-(D-Phe)-Arg-Trp-Asp-Arg-(D-Phe)-Gly-{X₂},
{X₁}-Tyr-Val-Met-Gly-His-Phe-(D-Arg)-Trp-Asp-Arg-(D-Phe)-Gly-{X₂},
{X₁}-Tyr-Val-Met-Gly-His-Phe-Arg-(D-Trp)-Asp-Arg-(D-Phe)-Gly-{X₂},
{X₁}-Tyr-Val-Met-Gly-His-(D-Phe)-Arg-(D-Trp)-Asp-Arg-(D-Phe)-Gly-{X₂},
{X₁}-Tyr-Val-Met-Gly-His-Nal-Arg-Trp-Asp-Arg-(D-Phe)-Gly-{X₂},
{X₁}-Tyr-Val-Met-Gly-His-(D-Nal)-Arg-Trp-Asp-Arg-(D-Phe)-Gly-{X₂},
{X₁}-Tyr-Val-Nle-Gly-His-Phe-Arg-Trp-Asp-Arg-Phe-Gly-{X₂},
{X₁}-Tyr-Val-Nle-Gly-His-(D-Phe)-Arg-Trp-Asp-Arg-Phe-Gly-{X₂},
{X₁}-Tyr-Val-Nle-Gly-His-Phe-(D-Arg)-Trp-Asp-Arg-Phe-Gly-{X₂},
{X₁}-Tyr-Val-Nle-Gly-His-Phe-Arg-(D-Trp)-Asp-Arg-Phe-Gly-{X₂},
{X₁}-Tyr-Val-Nle-Gly-His-(D-Phe)-Arg-(D-Trp)-Asp-Arg-Phe-Gly-{X₂},
{X₁}-Tyr-Val-Nle-Gly-His-Nal-Arg-Trp-Asp-Arg-Phe-Gly-{X₂},
{X₁}-Tyr-Val-Nle-Gly-His-(D-Nal)-Arg-Trp-Asp-Arg-Phe-Gly-{X₂},
{X₁}-Tyr-Val-Nle-Gly-His-Phe-Arg-Trp-Asp-Arg-(D-Phe)-Gly-{X₂},
{X₁}-Tyr-Val-Nle-Gly-His-(D-Phe)-Arg-Trp-Asp-Arg-(D-Phe)-Gly-{X₂},
{X₁}-Tyr-Val-Nle-Gly-His-Phe-(D-Arg)-Trp-Asp-Arg-(D-Phe)-Gly-{X₂},
{X₁}-Tyr-Val-Nle-Gly-His-Phe-Arg-(D-Trp)-Asp-Arg-(D-Phe)-Gly-{X₂},
{X₁}-Tyr-Val-Nle-Gly-His-(D-Phe)-Arg-(D-Trp)-Asp-Arg-(D-Phe)-Gly-{X₂},
{X₁}-Tyr-Val-Nle-Gly-His-Nal-Arg-Trp-Asp-Arg-(D-Phe)-Gly-{X₂}, and
{X₁}-Tyr-Val-Nle-Gly-His-(D-Nal)-Arg-Trp-Asp-Arg-(D-Phe)-Gly-{X₂},
wherein {X₁} is an optional linear amino acid probe consisting of from 2 to 20 consecutive amino acid residues covalently linked to the N-terminal Tyr,
wherein {X₂} is an optional linear amino acid probe consisting of from 2 to 20 consecutive amino acid residues covalently linked to the C-terminal Gly,
with the proviso that {X₁}, or {X₂}, or {X₁} and {X₂}, is present.

25. The peptide according to claim 1, wherein said carboxy terminal Gly is Glycine amide.

26. The peptide according to claim 1, wherein said peptide consists of a sequence selected from the group consisting of
{X₁}-Tyr-Val-Met-Gly-His-Phe-Arg-Trp-Asp-Arg-Phe-{X₂},
{X₁}-Tyr-Val-Met-Gly-His-(D-Phe)-Arg-Trp-Asp-Arg-Phe-{X₂},
{X₁}-Tyr-Val-Met-Gly-His-Phe-(D-Arg)-Trp-Asp-Arg-Phe-{X₂},
{X₁}-Tyr-Val-Met-Gly-His-Phe-Arg-(D-Trp)-Asp-Arg-Phe-{X₂},
{X₁}-Tyr-Val-Met-Gly-His-(D-Phe)-Arg-(D-Trp)-Asp-Arg-Phe-{X₂},
{X₁}-Tyr-Val-Met-Gly-His-Nal-Arg-Trp-Asp-Arg-Phe-{X₂},
{X₁}-Tyr-Val-Met-Gly-His-(D-Nal)-Arg-Trp-Asp-Arg-Phe-{X₂},
{X₁}-Tyr-Val-Met-Gly-His-Phe-Arg-Trp-Asp-Arg-(D-Phe)-{X₂},
{X₁}-Tyr-Val-Met-Gly-His-(D-Phe)-Arg-Trp-Asp-Arg-(D-Phe)-{X₂},
{X₁}-Tyr-Val-Met-Gly-His-Phe-(D-Arg)-Trp-Asp-Arg-(D-Phe)-{X₂},
{X₁}-Tyr-Val-Met-Gly-His-Phe-Arg-(D-Trp)-Asp-Arg-(D-Phe)-{X₂},
{X₁}-Tyr-Val-Met-Gly-His-(D-Phe)-Arg-(D-Trp)-Asp-Arg-(D-Phe)-{X₂},
{X₁}-Tyr-Val-Met-Gly-His-Nal-Arg-Trp-Asp-Arg-(D-Phe)-{X₂},
{X₁}-Tyr-Val-Met-Gly-His-(D-Nal)-Arg-Trp-Asp-Arg-(D-Phe)-{X₂},
{X₁}-Tyr-Val-Nle-Gly-His-Phe-Arg-Trp-Asp-Arg-Phe-{X₂},
{X₁}-Tyr-Val-Nle-Gly-His-(D-Phe)-Arg-Trp-Asp-Arg-Phe-{X₂},
{X₁}-Tyr-Val-Nle-Gly-His-Phe-(D-Arg)-Trp-Asp-Arg-Phe-{X₂},
{X₁}-Tyr-Val-Nle-Gly-His-Phe-Arg-(D-Trp)-Asp-Arg-Phe-{X₂},
{X₁}-Tyr-Val-Nle-Gly-His-(D-Phe)-Arg-(D-Trp)-Asp-Arg-Phe-{X₂},
{X₁}-Tyr-Val-Nle-Gly-His-Nal-Arg-Trp-Asp-Arg-Phe-{X₂},
{X₁}-Tyr-Val-Nle-Gly-His-(D-Nal)-Arg-Trp-Asp-Arg-Phe-{X₂},
{X₁}-Tyr-Val-Nle-Gly-His-Phe-Arg-Trp-Asp-Arg-(D-Phe)-{X₂},
{X₁}-Tyr-Val-Nle-Gly-His-(D-Phe)-Arg-Trp-Asp-Arg-(D-Phe)-{X₂},
{X₁}-Tyr-Val-Nle-Gly-His-Phe-(D-Arg)-Trp-Asp-Arg-(D-Phe)-{X₂},
{X₁}-Tyr-Val-Nle-Gly-His-Phe-Arg-(D-Trp)-Asp-Arg-(D-Phe)-{X₂},
{X₁}-Tyr-Val-Nle-Gly-His-(D-Phe)-Arg-(D-Trp)-Asp-Arg-(D-Phe)-{X₂},
{X₁}-Tyr-Val-Nle-Gly-His-Nal-Arg-Trp-Asp-Arg-(D-Phe)-{X₂}, and
{X₁}-Tyr-Val-Nle-Gly-His-(D-Nal)-Arg-Trp-Asp-Arg-(D-Phe)-{X₂},
wherein {X₁} is an optional linear amino acid probe consisting of from 2 to 20 consecutive amino acid residues covalently linked to the N-terminal Tyr,
wherein {X₂} is an optional linear amino acid probe consisting of from 2 to 20 consecutive amino acid residues covalently linked to the C-terminal Phe or (D-Phe),
with the proviso that {X₁}, or {X₂}, or {X₁} and {X₂}, is present.

27. The peptide according to claim 1, wherein said carboxy terminal Phe or (D-Phe) is a Phenylalanine amide.

28. A pharmaceutical composition comprising the peptide according to claim 1.

29. A method of treatment of a disease or condition in a subject in need thereof which comprises an effective amount of a peptide according to claim 1 wherein the disease or condition is an ischemic and/or inflammatory condition in the tissue of one or more organs of a mammal, wherein said mammal is a human (homo sapiens), wherein said organ is kidney, liver, brain, heart, muscles, bone marrow, skin, skeleton, lungs, the respiratory tract, spleen, exocrine glands, bladder, endocrine glands, reproduction organs including the fallopian tubes, eye, ear, vascular system, the gastroinstestinal tract including small intestines, colon, rectum, canalis analis or prostate gland, and wherein said ischemic and/or inflammatory condition is:
an acute ischemic and/or inflammatory condition, a subacute ischemic and/or inflammatory condition, a chronic ischemic and/or inflammatory condition, secondary ischemia, stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis; or
associated with surgery, major surgery, cardiothoracic surgery, abdominal surgery, surgery on the aorta and/or other major blood vessels, repair of one or more cardiac valves, cardiac artery bypass grafting (CABG), surgery on the aortic root or the aortic branch including the common carotic arteries, combined cardiac surgery, or valve(s) replacement and CABG and/or aortic root surgery; or
associated with organ transplantation, solid organ transplantation, heart transplantation, lung transplantation, combined heart and lung transplantation, liver transplantation or kidney transplantation; or
post-surgical systemic inflammatory response syndrome (SIRS), post-surgical organ dysfunction, post-surgical renal failure, acute kidney injury (AKI), neprotoxicity, or chronic renal failure (CRF); or
reperfusion injury, inflammatory disease, arthropathy (joint disease), rheumatoid arthritis (RA), gout, inflammatory diseases of the gastrointestinal system, or multiple sclerosis.


SEQUENCE LISTING (90 sequences)

SEQ ID NO: 1 - protein, 4 aa, Homo sapiens
HFRW

SEQ ID NO: 2 - protein, 4 aa, Homo sapiens
YVMG

SEQ ID NO: 3 - protein, 4 aa, Homo sapiens
KKKK

SEQ ID NO: 4 - protein, 5 aa, Homo sapiens
KKKKK

SEQ ID NO: 5 - protein, 6 aa, Homo sapiens
KKKKKK

SEQ ID NO: 6 - protein, 7 aa, Homo sapiens
KKKKKKK

SEQ ID NO: 7 - protein, 8 aa, Homo sapiens
KKKKKKKK

SEQ ID NO: 8 - protein, 9 aa, Homo sapiens
KKKKKKKKK

SEQ ID NO: 9 - protein, 10 aa, Homo sapiens
KKKKKKKKKK

SEQ ID NO: 10 - protein, 11 aa, Homo sapiens
KKKKKKKKKKK

SEQ ID NO: 11 - protein, 12 aa, Homo sapiens
KKKKKKKKKKKK

SEQ ID NO: 12 - protein, 13 aa, Homo sapiens
KKKKKKKKKKKKK

SEQ ID NO: 13 - protein, 14 aa, Homo sapiens
KKKKKKKKKKKKKK

SEQ ID NO: 14 - protein, 15 aa, Homo sapiens
KKKKKKKKKKKKKKK

SEQ ID NO: 15 - protein, 4 aa, Homo sapiens
EEEE

SEQ ID NO: 16 - protein, 5 aa, Homo sapiens
EEEEE

SEQ ID NO: 17 - protein, 6 aa, Homo sapiens
EEEEEE

SEQ ID NO: 18 - protein, 7 aa, Homo sapiens
EEEEEEE

SEQ ID NO: 19 - protein, 8 aa, Homo sapiens
EEEEEEEE

SEQ ID NO: 20 - protein, 9 aa, Homo sapiens
EEEEEEEEE

SEQ ID NO: 21 - protein, 10 aa, Homo sapiens
EEEEEEEEEE

SEQ ID NO: 22 - protein, 11 aa, Homo sapiens
EEEEEEEEEEE

SEQ ID NO: 23 - protein, 12 aa, Homo sapiens
EEEEEEEEEEEE

SEQ ID NO: 24 - protein, 13 aa, Homo sapiens
EEEEEEEEEEEEE

SEQ ID NO: 25 - protein, 14 aa, Homo sapiens
EEEEEEEEEEEEEE

SEQ ID NO: 26 - protein, 15 aa, Homo sapiens
EEEEEEEEEEEEEEE

SEQ ID NO: 27 - protein, 6 aa, Homo sapiens
EKKKKK

SEQ ID NO: 28 - protein, 6 aa, Homo sapiens
KEKKKK

SEQ ID NO: 29 - protein, 6 aa, Homo sapiens
KKEKKK

SEQ ID NO: 30 - protein, 6 aa, Homo sapiens
KKKEKK

SEQ ID NO: 31 - protein, 6 aa, Homo sapiens
KKKKEK

SEQ ID NO: 32 - protein, 6 aa, Homo sapiens
KKKKKE

SEQ ID NO: 33 - protein, 6 aa, Homo sapiens
EEKKKK

SEQ ID NO: 34 - protein, 6 aa, Homo sapiens
EKEKKK

SEQ ID NO: 35 - protein, 6 aa, Homo sapiens
EKKEKK

SEQ ID NO: 36 - protein, 6 aa, Homo sapiens
EKKKEK

SEQ ID NO: 37 - protein, 6 aa, Homo sapiens
EKKKKE

SEQ ID NO: 38 - protein, 6 aa, Homo sapiens
KEEKKK

SEQ ID NO: 39 - protein, 6 aa, Homo sapiens
KEKEKK

SEQ ID NO: 40 - protein, 6 aa, Homo sapiens
KEKKEK

SEQ ID NO: 41 - protein, 6 aa, Homo sapiens
KEKKKE

SEQ ID NO: 42 - protein, 6 aa, Homo sapiens
KKEEKK

SEQ ID NO: 43 - protein, 6 aa, Homo sapiens
KKEKEK

SEQ ID NO: 44 - protein, 6 aa, Homo sapiens
KKEKKE

SEQ ID NO: 45 - protein, 6 aa, Homo sapiens
KKKEEK

SEQ ID NO: 46 - protein, 6 aa, Homo sapiens
KKKEKE

SEQ ID NO: 47 - protein, 6 aa, Homo sapiens
KKKKEE

SEQ ID NO: 48 - protein, 6 aa, Homo sapiens
EEEKKK

SEQ ID NO: 49 - protein, 6 aa, Homo sapiens
EEKEKK

SEQ ID NO: 50 - protein, 6 aa, Homo sapiens
EEKKEK

SEQ ID NO: 51 - protein, 6 aa, Homo sapiens
EEKKKE

SEQ ID NO: 52 - protein, 6 aa, Homo sapiens
EKEEKK

SEQ ID NO: 53 - protein, 6 aa, Homo sapiens
EKEKEK

SEQ ID NO: 54 - protein, 6 aa, Homo sapiens
EKEKKE

SEQ ID NO: 55 - protein, 6 aa, Homo sapiens
EKKEEK

SEQ ID NO: 56 - protein, 6 aa, Homo sapiens
EKKEKE

SEQ ID NO: 57 - protein, 6 aa, Homo sapiens
EKKKEE

SEQ ID NO: 58 - protein, 6 aa, Homo sapiens
KKKEEE

SEQ ID NO: 59 - protein, 6 aa, Homo sapiens
KKEKEE

SEQ ID NO: 60 - protein, 6 aa, Homo sapiens
KKEEKE

SEQ ID NO: 61 - protein, 6 aa, Homo sapiens
KKEEEK

SEQ ID NO: 62 - protein, 6 aa, Homo sapiens
KEKKEE

SEQ ID NO: 63 - protein, 6 aa, Homo sapiens
KEKEKE

SEQ ID NO: 64 - protein, 6 aa, Homo sapiens
KEKEEK

SEQ ID NO: 65 - protein, 6 aa, Homo sapiens
KEEKKE

SEQ ID NO: 66 - protein, 6 aa, Homo sapiens
KEEKEK

SEQ ID NO: 67 - protein, 6 aa, Homo sapiens
KEEEKK

SEQ ID NO: 68 - protein, 6 aa, Homo sapiens
KKEEEE

SEQ ID NO: 69 - protein, 6 aa, Homo sapiens
KEKEEE

SEQ ID NO: 70 - protein, 6 aa, Homo sapiens
KEEKEE

SEQ ID NO: 71 - protein, 6 aa, Homo sapiens
KEEEKE

SEQ ID NO: 72 - protein, 6 aa, Homo sapiens
KEEEEK

SEQ ID NO: 73 - protein, 6 aa, Homo sapiens
EKKEEE

SEQ ID NO: 74 - protein, 6 aa, Homo sapiens
EKEKEE

SEQ ID NO: 75 - protein, 6 aa, Homo sapiens
EKEEKE

SEQ ID NO: 76 - protein, 5 aa, Homo sapiens
EKEEE

SEQ ID NO: 77 - protein, 6 aa, Homo sapiens
EEKKEE

SEQ ID NO: 78 - protein, 6 aa, Homo sapiens
EEKEKE

SEQ ID NO: 79 - protein, 6 aa, Homo sapiens
EEKEEK

SEQ ID NO: 80 - protein, 6 aa, Homo sapiens
EEEKKE

SEQ ID NO: 81 - protein, 6 aa, Homo sapiens
EEEKEK

SEQ ID NO: 82 - protein, 6 aa, Homo sapiens
EEEEKK

SEQ ID NO: 83 - protein, 6 aa, Homo sapiens
KEEEEE

SEQ ID NO: 84 - protein, 6 aa, Homo sapiens
EKEEEE

SEQ ID NO: 85 - protein, 6 aa, Homo sapiens
EEKEEE

SEQ ID NO: 86 - protein, 6 aa, Homo sapiens
EEEKEE

SEQ ID NO: 87 - protein, 6 aa, Homo sapiens
EEEEKE

SEQ ID NO: 88 - protein, 6 aa, Homo sapiens
EEEEEK

SEQ ID NO: 89 - protein, 12 aa, Homo sapiens
YVMGHFRWDRFG

SEQ ID NO: 90 - protein, 11 aa, Homo sapiens
YVMGHFRWDRF